(19) United States
(12) Patent Application Publication
(10) Pub. No.: US 2006/0121533 A1
(43) Pub. Date: Jun. 8, 2006

(54) ANALYSIS OF PROTEINS FROM BIOLOGICAL FLUIDS USING MASS SPECTROMETRIC IMMUNOASSAY

(75) Inventors: Urban A. Kiernan, Gilbert, AZ (US); Eric Niederkofler, Glendale, AZ (US); Kemmons A. Tubbs, Mesa, AZ (US); Dobrin Nedelkov, Tempe, AZ (US); Randall W. Nelson, Phoenix, AZ (US)

Correspondence Address: SNELL & WILMER;ONE ARIZONA CENTER, 400 EAST VAN BUREN, PHOENIX, AZ, 850040001, US

(73) Assignee: INTRINSIC BIOPROBES, INC., Tempe, AZ (US)

(21) Appl. No.: 11/163,587

(22) Filed: Oct. 24, 2005

Related U.S. Application Data
(62) Division of application No. 10/188,178, filed on Jul. 2, 2002.
(60) Provisional application No. 60/302,640, filed on Jul. 2, 2001.
(60) Provisional application No. 60/306,957, filed on Jul. 21, 2001.

(51) Int. Cl. G01N 33/53; G01N 33/00

(52) U.S. Cl. 435/7.1; 436/86

(57) ABSTRACT

Presented herein are methods, devices and kits for the mass spectrometric immunoassay (MSIA) of proteins present in complex biological fluids or extracts. Pipettor tips containing porous solid supports that are covalently derivatized with affinity ligand and used to extract specific proteins and their variants from various biological fluids. Nonspecifically bound compounds are rinsed from the extraction devices using a series of buffer and water rinses, after which the wild type protein (and/or its variants) are eluted directly onto a target in preparation for analysis such as matrix-assisted laser desorption/ionization time-of-flight mass spectrometry (MALDI-TOF MS). Mass spectrometry of the eluted sample then follows with the retained proteins identified via accurate molecular mass determination. Protein and variant levels can be determined using quantitative methods in which the protein/variant signals are normalized to signals of internal reference standard species (either doped into the samples prior to the MSIA analysis, or other endogenous protein co-extracted with the target proteins) and the values compared to a working curves constructed from samples containing known concentrations of the protein or variants. Such MSIA devices, kits and methods have significant application in the fields of; basic research and development, proteomics, protein structural characterization, drug discovery, drug-target discovery, therapeutic monitoring, clinical monitoring and diagnostics, as well as in the high throughput screening of large populations to establish and recognize protein/variant patterns that are able to differentiate healthy from diseased states.

### CROSS-REFERENCE TO RELATED APPLICATIONS

This application is a divisional of and claims benefit of priority to U.S. Non-Provisional patent application Ser. No. 10/188,178, filed on Jul. 2, 2002, and also entitled “Analysis of Proteins from Biological Fluids Using Mass Spectrometric Immunoassay”, which application claims the benefit of, and priority to, provisional application Ser. No. 60/302,640, filed Jul. 2, 2001 and provisional application Ser. No. 60/306,957, filed Jul. 20, 2001, which applications are hereby incorporated by reference in their entirety.

### FIELD OF INVENTION

The present invention relates to devices, kits and methods for the rapid characterization of biomolecules recovered directly from biological samples. The devices, kits and methods according to the present invention summarily provide the basis for mass spectrometric immunoassays (MSIA), which are able to qualitatively and quantitatively analyze specific proteins, and their variants, present in a variety of biological fluids and extracts. Such MSIA devices, kits and methods have significant application in the fields of; basic research and development, proteomics, protein structural characterization, drug discovery, drug-target discovery, therapeutic monitoring, clinical monitoring and diagnostics, as well as in the high throughput screening of large populations to establish and recognize protein/variant patterns that are able to differentiate healthy from diseased states.

### BACKGROUND OF INVENTION

With the recent first draft completion of the human genome, much attention is now shifting to the field of proteomics, where gene products (proteins), their variants, interacting partners and the dynamics of their regulation and processing are the emphasis of study. Such studies are essential in understanding, for example, the mechanisms behind genetic/environmentally induced disorders or the influences of drug mediated therapies, as well as potentially becoming the underlying foundation for further clinical and diagnostic analyses. Critical to these studies is the ability to qualitatively determine specific variants of whole proteins (i.e., splice variants, point mutations, posttranslationally modified versions, and environmentally/therapeutically-induced modifications) and the ability to view their quantitative modulation. Moreover, it is becoming increasing important to perform these analyses from not just one, but multiple biological fluids/extracts derived from a single individual.

Accordingly, there is a pressing need for rapid, sensitive and accurate analytical approaches for the analysis of proteins and their variants. This present application considers the proteins; urinary protein 1 (UP1), IgG light chains kappa and lambda (referred to as Bence Jones Proteins (BJP)), insulin-like growth factor (IGF-1), serum amyloid A (SAA), vitamin D binding protein (VDB), leptin (LEP), Tamm Horsfall Glycoprotein (THG), albumin (ALB), lysozyme (LYC), a-defensins (HNP), immunoglobulin (IgG), apolipoprotein E (ApoE), apolipoprotein AII (ApoA-II), apolipoprotein AI (ApoA-I), C-reactive protein (CRP), serum amyloid P component (SAP), cystatin C (CYTC), transthyretin (TTR), transferrin (TRFE), and retinol binding protein (RBP) present in various biological fluids/extracts found in individuals (humans).

There are several challenges inherent to the analysis of these proteins, or for that matter, all proteins in general. The greatest challenge is the fact that any protein considered relevant enough to be analyzed resides in vivo in a complex biological environment or media. The complexity of these biological media present a challenge in that, oftentimes, a protein of interest is present in the media at relatively low levels and is essentially masked from analysis by a large abundance of other biomolecules, e.g., proteins, nucleic acids, carbohydrates, lipids and the like. In other instances, (e.g., the lipoproteins), proteins are complexed tightly with other biomolecules that might interfere with their analysis. In order to analyze proteins of interest from—and in—their native environment, assays capable of assessing proteins present in a variety of biological fluids and/or extracts, both qualitatively and quantitatively, are needed. These assays must: 1) be able to selectively retrieve and concentrate specific proteins/biomarkers from various biological fluid/extract for subsequent high-performance analyses, 2) be able to quantify targeted proteins, 3) be able to recognize variants of targeted proteins (e.g., splice variants, point mutations, posttranslational modifications and environmentally /therapeutically induced chemical modifications) and to elucidate their nature, 4) be capable of analyzing for, and identifying, ligands interacting with targeted proteins, and 5) be able to analyze the same protein from multiple fluids/extracts taken from a single individual. Moreover, it is great value to apply such analyses in high throughput to large numbers of samples in order to determine a statistical “normal” profile for any given protein in any particular fluid/extract from which “abnormal” differences are readily recognizable. Causes of such abnormalities may be related to genetic makeup, disease, therapeutic treatments or environmental stresses.

In order to accomplish such assays, it is necessary to combine selective purification/concentration approaches with analytical techniques capable of the rigorous structural characterization of biomolecules. One such approach is mass spectrometric immunoassay (MSIA), where affinity isolation is used in combination with matrix-assisted laser desorption/ionization time-of-flight mass spectrometry (MALDI-TOF MS) to form a concerted, high-performance technique for the analysis of proteins [Nelson et al, Anal. Chem 1995]. Utilizing the approach, a single pan-antibody can be used to retrieve all variants of a specific protein from a biological fluid, upon which each variant is detected during mass spectrometry at a unique and characteristic molecular mass. Moreover, resolution of related species also allows mass-shifted variants of a target protein to be intentionally incorporated into the analysis for use as internal reference standard (IRS) for quantitative analysis. Applied differently, the inherent resolution of MALDI-TOF MS allows assays to be devised using multiple affinity ligands to selectively purify/concentrate and then analyze multiple proteins in a single assay. Overall, the MSIA approach can be used for the unambiguous detection and rigorous quantification of proteins and variants retrieved from complex biological systems. To date, however, approaches such as MSIA have not been driven in the breadth or capacity needed to make a significant impact in the biological sciences. Specifically, devices, kits and methods for the analysis of large numbers of selected proteins present in multiple biological fluids/extracts (in large numbers of individuals) are lacking.

For these foregoing reasons, there is a driven need for MSIA devices, kits and methods for the rapid and efficient analysis of the above-mentioned proteins and other specific proteins and variants present in various biological fluids. Moreover, there is a need to correlate the results of such analyses with disease states in order to employ empirical findings in further applications such as drug and drug-target discovery, clinical monitoring and diagnostics.

All publications and patent applications listing Randall W. Nelson as inventor or author are herein incorporated by reference to the same extent as if each individual publication or patent application was specifically and individually indicated to be incorporated by reference. Although the present invention has been described in some detail by way of illustration and example for purposes of clarity and understanding, it will be apparent that certain changes and modifications may be practiced within the scope of the appended claims.

### SUMMARY OF INVENTION

It is an object of the present invention to devise MSIA methods that are able to prepare micro-samples for mass spectrometry directly from biological fluid.

It is another object of the present invention to construct pipettor tips (termed MSIA-Tips) containing porous solid supports that are constructed, covalently derivatized with affinity ligand, and used to extract specific proteins and their variants in preparation for mass spectrometry.

Yet another object of the present invention is to apply in use the aforementioned MSIA methods and devices in analyzing specific proteins and their variants from biological fluids and extracts.

Another object of the present invention is to provide for protein and variant quantification using MSIA by ensuring the presence of a second protein species in the assay to serve as an IRS.

It is yet another object of the present invention to provide MSIA assays that have adequate quantitative dynamic ranges, accuracies, and linearities to cover the concentrations of proteins expected in the biological fluids.

A further object of this present invention is to provide useful product kits for the detection, qualification, and quantification of specific proteins and variants present in a variety of biological fluids or extracts obtained from a single individual.

It is still another object of the present invention to devise MSIA product kits for the analysis of the following proteins, and their variants, present in various biological fluids/extracts found in individuals (humans): urinary protein 1 (UP1), IgG light chains kappa and lambda (referred to as Bence Jones Proteins (BJP)), insulin-like growth factor (IGF-1), serum amyloid A (SAA), vitamin D binding protein (VDB), leptin (LEP), Tamm Horsfall Glycoprotein (THG), albumin (ALB), lysozyme (LYC), a-defensins (HNP), immunoglobulin (IgG), apolipoprotein E (ApoE), apolipoprotein AII (ApoA-II), apolipoprotein Al (ApoA-I), C-reactive protein (CRP), serum amyloid P component (SAP), cystatin C (CYTC), transthyretin (TTR), transferrin (TRFE), and retinol binding protein (RBP).

Yet a further object of the present invention is to use the aforementioned kits, devices and methods to detect variants of the target proteins.

Another object of the present invention is to use the methods, devices and kits in the fields of basic research and development, proteomics, protein structural characterization, drug discovery, drug-target discovery, therapeutic monitoring, clinical monitoring and diagnostics.

It is still a further objective of the present invention to use the MSIA kits, devices and methods in general population screens, which include both diseased and healthy-state individuals, to recognize and establish protein and variant patterns that correlate with disease.

The present invention includes the ability to selectively retrieve and concentrate specific biomolecules from biological fluid for subsequent high-performance analyses (e.g. MALDI-TOF MS), the ability to identify targeted biomolecules, the ability to quantify targeted biomolecules, the ability to recognize variants of targeted biomolecules (e.g., splice variants, point mutations, posttranslational modifications, and environmentally/therapeutically induced chemical modifications) and to elucidate their nature, and the capability to analyze for, and identify, ligands interacting with targeted biomolecules. The novel features that are considered characteristic of the invention are set forth with particularity in the appended claims. The invention itself, however, both as to its structure and its operation together with the additional objects and advantages thereof will best be understood from the following description of the preferred embodiment of the present invention when read in conjunction with the accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### BRIEF DESCRIPTION OF THE DRAWINGS

FIG. 1 is a schematic illustration of the MSIA procedure.

FIG. 2 is an illustration of MSIA analysis of cystatin C (CYTC) from human plasma.

FIG. 3 is an illustration of MSIA analysis of cystatin C (CYTC) from human urine.

FIG. 4 is an illustration of MSIA analysis of cystatin C (CYTC) from human tears.

FIG. 5 is an illustration of MSIA analysis of cystatin C (CYTC) from human saliva.

FIG. 6 is an illustration of MSIA analysis of cystatin C (CYTC) from human nasal mucus.

FIG. 7 is an illustration of MSIA analysis of vitamin D binding protein (VDB) from human plasma.

FIG. 8 is an illustration of MSIA analysis of vitamin D binding protein (VDB) from human urine.

FIG. 9 is an illustration of MSIA analysis of urine protein 1 (UP1) from human plasma.

FIG. 10 is an illustration of MSIA analysis of urine protein 1 (UP1) from human urine.

FIG. 11 is an illustration of MSIA analysis of Bence-Jones kappa (BJ-k) from human urine.

FIG. 12 is an illustration of MSIA analysis of Bence-Jones lambda (BJ-L) from human urine.

FIG. 13 is an illustration of MSIA analysis of insulin like growth factor 1 (IGF-1) from human plasma.

FIG. 14 is an illustration of MSIA analysis of serum amyloid A (SAA) from human plasma.

FIG. 15 is an illustration of MSIA analysis of human leptin (LEP) from human plasma.

FIG. 16 is an illustration of MSIA analysis of Tamm-Horsfall glycoprotein (THG) from human urine.

FIG. 17 is an illustration of MSIA analysis of albumin (ALB) from human urine.

FIG. 18 is an illustration of MSIA analysis of lysozyme C (LYC) from human plasma.

FIG. 19 is an illustration of MSIA analysis of lysozyme C (LYC) from human urine.

FIG. 20 is an illustration of MSIA analysis of lysozyme C (LYC) from human saliva.

FIG. 21 is an illustration of MSIA analysis of α-defensins (HNP) from human urine.

FIG. 22 is an illustration of MSIA analysis of α-defensins (HNP) from human saliva.

FIG. 23 is an illustration of MSIA analysis of immunoglobulin G (IgG) from human urine.

FIG. 24 is an illustration of MSIA analysis of serum amyloid P component (SAP) from human plasma.

FIG. 25 is an illustration of MSIA analysis of serum amyloid P component (SAP) from human urine.

FIG. 26 is an illustration of MSIA analysis of retinol binding protein (RBP) from human plasma.

FIG. 27 is an illustration of MSIA analysis of retinol binding protein (RBP) from human urine.

FIG. 28 is an illustration of MSIA analysis of C-reactive protein (CRP) from human plasma.

FIG. 29 is an illustration of MSIA analysis of C-reactive protein (CRP) from human urine.

FIG. 30 is an illustration of MSIA multi-analysis of RBP, CRP and SAP from human plasma.

FIG. 31 is a calibration curve generated by the MSIA multi-analysis of human plasma samples undergone purified CRP standard addition.

FIG. 32 is a histogram of high throughput MSIA multi-analysis of RBP, CRP and SAP comparing 96 human plasma samples.

FIG. 33 is an illustration of MSIA analysis of transthyretin (TTR) from human plasma.

FIG. 34 is an illustration of MSIA analysis of transthyretin (TTR) from human urine.

FIG. 35 is an illustration of MSIA analysis of transferrin (TRFE) from human plasma.

FIG. 36 is an illustration of MSIA analysis of transferrin (TRFE) from human urine.

FIG. 37 is an illustration of MSIA analysis of apolipoprotein E (ApoE) from human plasma.

FIG. 38 is an illustration of MSIA analysis of apolipoprotein A-I (ApoA-I) from human plasma.

FIG. 39 is an illustration of MSIA analysis of apolipoprotein A-II (ApoA-II) from human plasma.

FIG. 40 is an illustration of MSIA analysis of biotinylated peptide from human plasma by use of avidin-MSIA-Tip.

FIG. 41 is an illustration of MSIA analysis of biotinylated peptide from human urine by use of avidin-MSIA-Tip.

FIGS. 42a-42d are illustrations of MSIA analysis of a His-tagged recombinant protein from E. coli lysate using and comparing anti His-tagged antibody MSIA-Tips with NTA chelator MSIA-Tips.

### DETAILED DESCRIPTION

The present invention provides for methods, devices and kits for the MSIA analysis of specific proteins and variants present in various biological fluids and extracts. Such analyses are essential for the detailed, full-length characterization of proteins as a function of the biological fluid/extract from which they originate. Proteins of concern in the present invention are urinary protein 1 (UP1), IgG light chains kappa and lambda (referred to as Bence Jones Proteins (BJP)), insulin-like growth factor (IGF-1), serum amyloid A (SAA), vitamin D binding protein (VDB), leptin (LEP), Tamm Horsfall Glycoprotein (THG), albumin (ALB), lysozyme (LYC), a-defensins (HNP), immunoglobulin (IgG), apolipoprotein E (ApoE), apolipoprotein AII (ApoA-II), apolipoprotein AI (ApoA-I), C-reactive protein (CRP), serum amyloid P component (SAP), cystatin C (CYTC), transthyretin (TTR), transferrin (TRFE), and retinol binding protein (RBP).

Another embodiment of the present invention provides for methods used in the quantification of proteins and variants present in various biological fluids. In certain analyses, multiple proteins/variants were simultaneously retrieved from a given biological fluid/extract. The target proteins were then quantified (either relative or absolute) by equating relative signal intensities/integrals with analyte concentration.

Yet another embodiment of the present invention provides for the use of MSIA in screening of individuals in large populations for specific proteins and variants present in various biological fluids. When applied to multiple individuals, the MSIA assays are able to yield intra-individual and inter-individual details, regarding a specified protein, which upon correlation can be linked to genetic, transcriptional or posttranslational differences, disease state, response to therapy or environmental stress, or differences in metabolism/catabolism.

In another embodiment, the present invention is used to discover new protein variants that are linked to either healthy or disease states. During analyses, different variants of the target proteins are observed dependent on the biological fluid or individual from which they were retrieved. The differences observed using the MSIA approach form the basis for clinical or diagnostic assays.

Specific embodiments in accordance with the present invention will now be described in detail using the following lexicon. These examples are intended to be illustrative, and the invention is not limited to the materials, methods or apparatus set forth in these embodiments.

As used herein, “MSIA-Tips” refers to a pipettor tip containing an affinity reagent.

As used herein, “affinity ligand” refers to atomic or molecular species having an affinity towards analytes present in biological mixtures. Affinity ligands may be organic, inorganic or biological by nature, and can exhibit broad (targeting numerous analytes) to narrow (target a single analyte) specificity. Examples of affinity ligands include, but are not limited to, receptors, antibodies, antibody fragments, synthetic paratopes, enzymes, proteins, multi-subunit protein receptors, mimics, chelators, nucleic acids, and aptamers.

As used herein, “analyte” refers to molecules of interest present in a biological sample. Analytes may be, but are not limited to, nucleic acids, DNA, RNA, peptides, polypeptides, proteins, antibodies, protein complexes, carbohydrates or small inorganic or organic molecules having biological function. Analytes may naturally contain sequences, motifs or groups recognized by the affinity ligand or may have these recognition moieties introduced into them via chemical or enzymatic processes.

As used herein, “biological fluid or extract” refers to a fluid or extract having a biological origin. Biological fluid may be, but are not limited to, cell extracts, nuclear extracts, cell lysates or biological products used to induce immunity or substances of biological origin such as excretions, blood, sera, plasma, urine, sputum, tears, feces, saliva, membrane extracts, and the like.

As used herein, “internal reference standard” refers to analyte species that are modified (either naturally or intentionally) to result in a molecular weight shift from targeted analytes and their variants. The IRS can be endogenous in the biological fluid or introduced intentionally. The purpose of the IRS is that of normalizing all extraction, rinsing, elution and mass spectrometric steps for the purpose of quantifying targeted analytes and/or variants.

As used herein, “posttranslational modification” refers to any alteration that occurs after synthesis of the polypeptide chain. Posttranslational modifications may be, but are not limited to, glycosylation, phosphorylation, sulphation, amidation, cysteinylation, dimerization, or enzymatic or chemical additions or cleavages. The cause of the posttranslational modifications can be endogenous (e.g., systematic within the individual), environmentally or therapeutically induced or in response to external stimuli such as stress or infection (e.g., bacterial or viral).

As used herein, “genetic difference” refers to differences in nucleic acid sequence (e.g., DNA or RNA) that result in a recognizable mass shift on the protein level. Genetic differences may be nucleotide polymorphisms, variations in short tandem repeats, variations in allele, or transcriptional variations (e.g., splice variants).

As used herein, “wild-type” refers to the variation of a given protein most commonly found in nature. The wild-type protein is generally recognized as the functional form of the protein, including all transcriptional and posttranslational processing. The wild-type protein can be found empirically using MSIA by assaying large numbers of individuals and determining the high-percentage variant.

As used herein, “variant” refers to different forms of a given proteins resulting from genetic differences or posttranslational modifications. As generally applied, MSIA recognizes the variants by observing them as signals mass-shifted from those expected for the wild-type protein.

As used herein, “mass spectrometer” refers to a device able to volatilize/ionize analytes to form vapor-phase ions and determine their absolute or relative molecular masses. Suitable forms of volatilization/ionization are laser/light, thermal, electrical, atomized/sprayed and the like or combinations thereof. Suitable forms of mass spectrometry include, but are not limited to, Matrix Assisted Laser Desorption/Time of Flight Mass Spectrometry (MALDI-TOF MS), electrospray (or nanospray) ionization (ESI) mass spectrometry, or the like or combinations thereof.

### EXAMPLE 1

### General MSIA Method

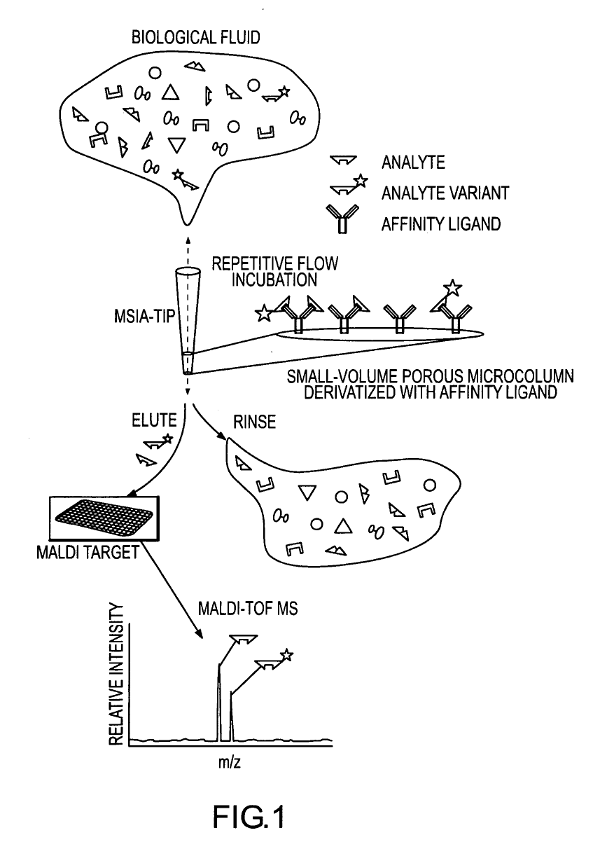

The general MSIA approach is shown graphically in FIG. 1. MSIA-Tips, containing porous solid supports covalently derivatized with affinity ligands that are used to extract the specific analytes and their variants from biological samples by repetitively flowing the samples through the MSIA-Tips. Once washed of non-specifically bound compounds, the retained analytes are eluted onto a mass spectrometer target using a MALDI matrix. MALDI-TOF MS then follows, with analytes detected at precise m/z values. The analyses are qualitative by nature but can be made quantitative by incorporating mass-shifted variants of the analyte into the procedure for use as internal standards.

With regard to the proteins listed in the following Examples, mass spectrometric immunoassays were performed in the following general manner (additional methodologies specific to each protein are addressed in the Examples):

The MSIA-Tips used in urine and blood analyses were construct having a single-piece (monolithic—acting both a stationary phase and derivatizable support) porous micro-frit (0.25-2.5 μL dead volume) at the entrance to a microcolumn with adequate volume (10-1000 μL) to accommodate the volume of the sample. The microcolumn barrels were constructed from glass or plastic and in the form of tapered or straight capillaries or pipettor tips. The porous microfrits were manufactured using any number of derivatization schemes that ultimately result in free functional groups able to be activated for subsequent coupling of antibodies/affinity ligands via covalent linkage through amines, carboxylic acids or sulfhydryls. Antibodies were monoclonal or polyclonal and were prepared from the serum of inoculated organism (e.g., rabbit, mouse, goat or other antibody-producing organism) or ascites fluid via Protein A/G extraction of affinity purification towards the antigen prior to linkage to the MSIA-Tips. Other affinity ligands were isolated/prepared using similar affinity and standard chromatographic approaches.

For analysis from blood, a 50 μL sample of human whole blood was collected from a lancet-punctured finger using a capillary microcolumn (heparinized, EDTA or no coating) and mixed with 200 μL of HBS buffer (10 mM HEPES, 150 mM NaCl, pH 7.4, 3 mM EDTA, 0.005% polysorbate 20 (v/v)) and centrifuged for 30 seconds (at 7,000 RPM, 3000×g) to pellet the red blood cells. Aliquots (10-220 μL) of the supernatant (diluted plasma) were subsequently mixed with additional HBS buffer to bring the total volume of the diluted plasma to 400-1200 μL. Analyses were performed from a diluted plasma sample by repeatedly (5-500 cycles, 20-200 μL/cycle, 10-100 cycles/minute) drawing and expelling the sample on antibody-derivatized affinity microcolumns (MSIA-Tips). After selective extraction/concentration of the specified protein, tips were rinsed (with e.g., water, buffers, detergents, organic solvents or combinations thereof) to remove trace non-specifically retained compounds. Retained compounds were eluted for MALDI-TOF MS using a small volume (0.5-5 μL) of a chaotrope and then adding a common MALDI matrix solution (e.g., α-cyano-4-hydroxycinnamic acid or sinapinic acid in acetonitrile/water/trifluoroacetic acid mixture) or simply by using a MALDI matrix solution. MALDI-TOF MS was performed using linear delayed-extraction mass spectrometer, although other forms of MALDI mass spectrometers could be used.

Oftentimes, multiple MSIA analyses were performed serially from a single plasma sample by addressing the sample with a first antibody-derivatized MSIA-Tips followed by subsequent tips (e.g., a second tip specific to a second protein, a third tip specific to a third protein . . . ). This approach increased the efficiency of use of a single sample and resulted in the need to draw less blood from an individual.

Analyses were performed from urine using an approach similar to that described for blood plasma. Urine samples were prepared for analysis by addition of a pH compensating buffer such as 2M ammonium acetate (pH=7.6) that contained a protease inhibitor cocktail. Additionally, because of its availability, and generally lower concentration of target proteins, larger volumes (0.2-50 mL) of urine were addressed. To ensure complete incubation of the larger volumes with the MSIA-Tips, a larger number of incubation cycles (100-1000) were used. Rinse, elution, preparation and MALDI-TOF MS protocols were the same as for plasma analyses.

Oftentimes, multiple proteins were analyzed from a single urine sample in parallel by addressing the sample with parallel repeating robotics fitted with multiple MSIA-Tips, each targeting a different protein. This approach required less time spent for each analysis, as well as made more efficient use of a sample.

### EXAMPLE 2

### Cystatin C

Cystatin C (CYTC) is an extracellular cysteine protease inhibitor that has been indicated as a putative biomarker for a number of inflammatory ailments. CYTC plasma levels can be used reliably as a measure of glomerular filtration rate, which has been linked to renal failure. A cystatin variant caused by a T→A point mutation (replacing leucine with glutamine) is a cause of Icelandic hereditary cystatin C amyloid angiopathy, an autosomal dominant disorder characterized by amyloid deposition of the CYTC variant in almost all tissues. A number of carcinoma cell lines have been reported to secrete CYTC, leading to investigations of its role as a possible tumor marker. In addition, it has been shown that urinary concentration of CYTC is greatly increased in patients with tubular disorders.

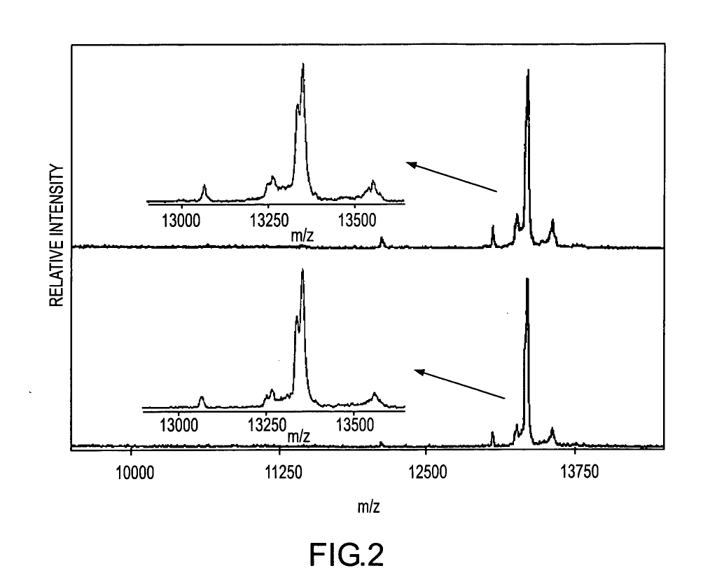

With reference to FIG. 2, a MSIA analysis of cystatin C (CYTC) from human plasma sample was performed. Two healthy individuals were analyzed in the following manner. 50 μL samples of human whole blood were collected from a lancet-punctured finger using a heparinized microcolumn, mixed with 200 μL HBS buffer and centrifuged for 30 seconds (at 7,000 RPM, 3000×g) to pellet the red blood cells. A 15 μL of each supernatant was mixed with 135 μL of HBS buffer (10 mM HEPES, 150 mM NaCl, pH 7.4, 3 mM EDTA, 0.005% polysorbate 20 (v/v)), yielding a 1:100 total dilution of the human plasma (plasma constitutes 50% of the whole blood). Polyclonal anti-CYTC MSIA-Tips were made via 1,1′-Carbonyidiimidazole (CDI)-mediated coupling of anti-CYTC antibody to carboxymethyidextran (CMD) modified MSIA-Tips. The diluted plasma solutions were repetitively (50 times, 100 μL each time) aspired and dispensed through the anti-CYTC MSIA-Tips. A rinse with HBS (10 aspirations and dispensing, 100 μL each, performed twice) and water (10×100 μL, twice) followed. The captured proteins were eluted from the MSIA-Tip with a small volume of MALDI matrix (saturated aqueous solution of sinapinic acid (SA), in 33% (v/v) acetonitrile, 10% (v/v) acetone, 0.4% (v/v) trifluoroacetic acid) and stamped onto a MALDI target array surface comprised of self-assembled monolayers chemically masked to make hydrophilic/hydrophobic contrast target arrays. The sample spots on the target array were analyzed using MALDI-TOF mass spectrometry. The resulting mass spectra are shown in FIG. 2. Signals due to the singly-charged ion of CYTC are observed, along with a doubly charged CYTC signals. Interestingly, the CYTC signal is in fact a doublet of peaks (inset, FIG. 2) resulting from the partial hydroxylation of a proline residue at position 3. In addition, multiple N-terminal truncated forms of CYTC are observed. The MSIA analysis of plasma CYTC can be used for population screening of genetic mutations as well as assess renal function.

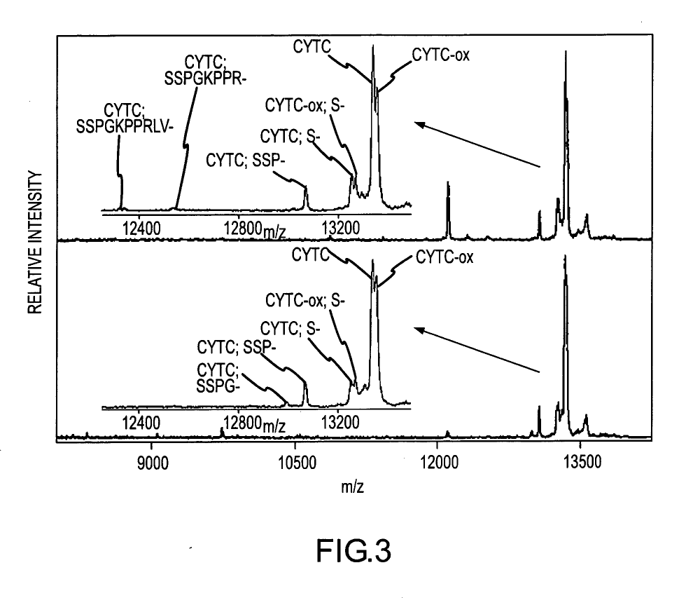

With reference to FIG. 3, MSIA analyses were performed to analyze CYTC present in the urine of the two individuals. 30 mL samples of human urine (fresh, mid-stream voids) were collected and mixed with 30 mL HBS buffer (1:1 ratio showing) in larger plastic containers. Polyclonal anti-CYTC MSIA-Tips were made in the same fashion as described above. The entire 60 mL of the 1:1 diluted urine solution was used as a sample and was repetitively (300 times, 200 μL each time) aspired and dispensed through the anti-CYTC MSIA-Tip. A rinse with HBS (10 aspirations and dispensing, 200 μL each) and water (10×200 μL) followed. The captured proteins were eluted from the MSIA-Tip with a small volume of MALDI matrix (saturated aqueous solution of sinapinic (SA), in 33% (v/v) acetonitrile, 10% (v/v) acetone, 0.4% (v/v) trifluoroacetic acid) and stamped onto a MALDI target array surface comprised of self-assembled monolayers chemically masked to make hydrophilic/hydrophobic contrast target arrays. The sample spots on the target array were analyzed using MALDI-TOF mass spectrometry. The resulting mass spectra are shown in FIG. 3. Signals due to the singly-charged ion of CYTC are observed. As in the previous figure, the CYTC signals are comprised of two closely spaced signals (see the expanded region inset). Multiple N-truncated truncated versions of CYTC are also observed, similar to plasma, when retrieved from urine. Some of these variants are found to be unique to the urine profile of CYTC, and serve as a point of reference in differentiating healthy from diseased states. This assay may also be able to screen for genetic variations manifested in the protein. The MSIA analyses of urinary CYTC is capable of screening populations for genetic variants as well as assess kidney function.

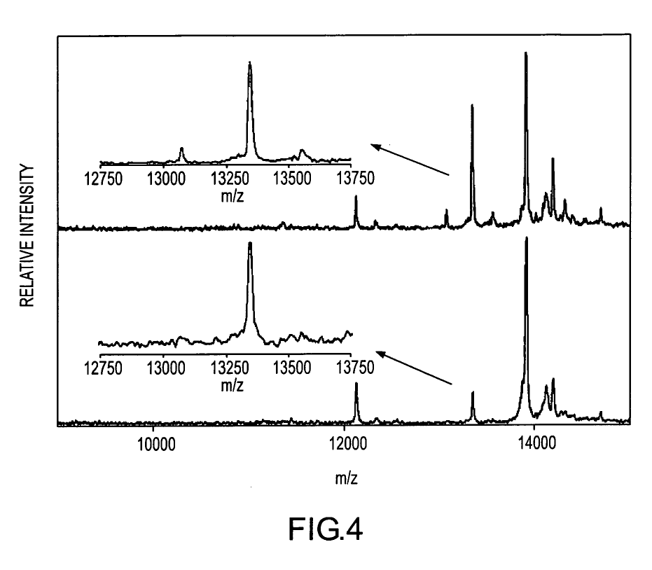

FIG. 4 shows MSIA spectra of CYTC analyzed from human tears. The protocols employed in the analysis were the same as those described for the plasma assay, exception using ˜10 uL of tear fluid instead of plasma. Analyses were performed for the same individuals participating in the study. The CYTC profile from tears is different than that of plasma or urine by the presence of only a single main peak instead of a doublet and the absence of truncated variants.

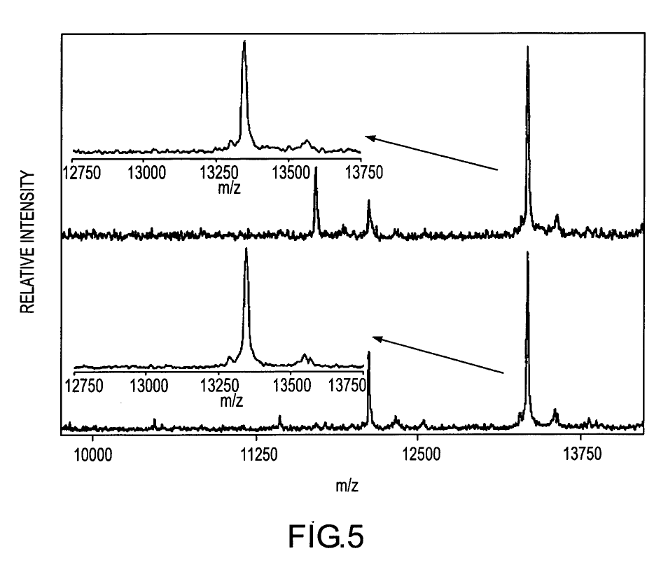

FIG. 5 shows MSIA spectra of CYTC analyzed from human saliva. The protocols employed in the analysis were the same as those described for the plasma assay, exception using ˜2 uL of whole saliva instead of plasma. Analyses were performed for the same individuals participating in the study. The CYTC profile of saliva is similar to that of tears by the absence of truncated products and peak doublet.

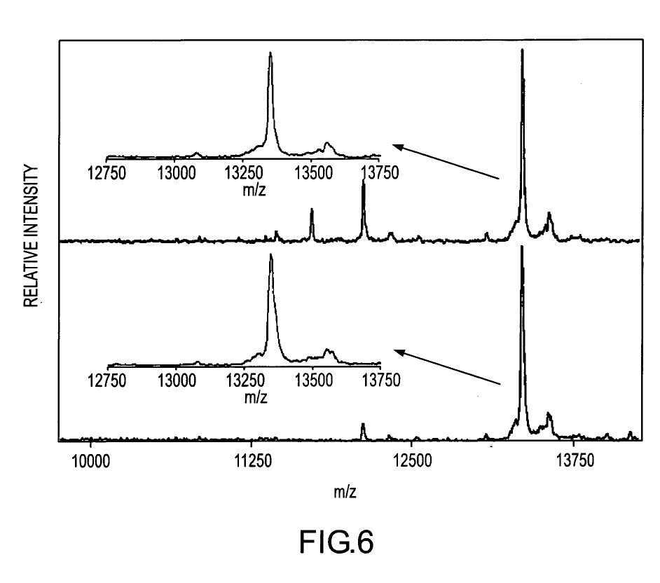

FIG. 6 shows MSIA spectra of CYTC analyzed from human nasal mucus. The protocols employed in the analysis were the same as those described for the plasma assay, exception using ˜1 uL of mucus instead of plasma. Analyses were performed for the same individuals participating in the study. CYTC profiles were similar to those found in saliva and tears.

Collectively, these examples illustrate the utility of MSIA in the analysis of proteins and variants both intra- and inter-individually. Specifically, analysis of CYTC from different biological fluids produced recognizably different profiles. Consistency between the profiles obtained from any one biofluid lays the foundation (i.e., repetitive, predictable results) from which differences related to stimuli (e.g., disease, therapy, environmental stress) can be judged. In this manner, the MSIA approach is utilized as a discovery platform, which later can be used in screening individuals for clinical and diagnostic purposes.

### EXAMPLE 3

### Vitamin D Binding Protein

Vitamin D binding protein, VDB (also known as group specific component (Gc) or GC-globulin), is a 52 kDa multifunctional protein found in plasma, urine, and other bodily fluids. The concentration of VDB in plasma is ˜300 82 g/L. Over 120 variants of VDB have been identified, with three alleles being dominantly present. VDB has a connotation as a cancer biomarker. Namely, cancerous cells secrete the enzyme alpha-N-acetylgalactosaminidase into the bloodstream, which completely deglycosilates VDB and thus prevents its conversion into the macrophage activating factor (the conversion is achieved by removal of a β-galactose and sialic acid from the VDB trisacharide glycan, leaving N-acetyl-galactosamine (GaINAc) still bound to Asp288). Removal of the residual GaINAc by this enzyme, which was recently found to be exclusively responsible for deglycosylation of VDB, prevents the VDB conversion into the macrophage-activating factor. Since the alpha-N-acetylgalactosaminidase activity in the blood stream can be used as diagnostic/prognostic value of cancer, by assaying the deglycosylated VDB directly from plasma, the presence of the enzyme can be indirectly determined.

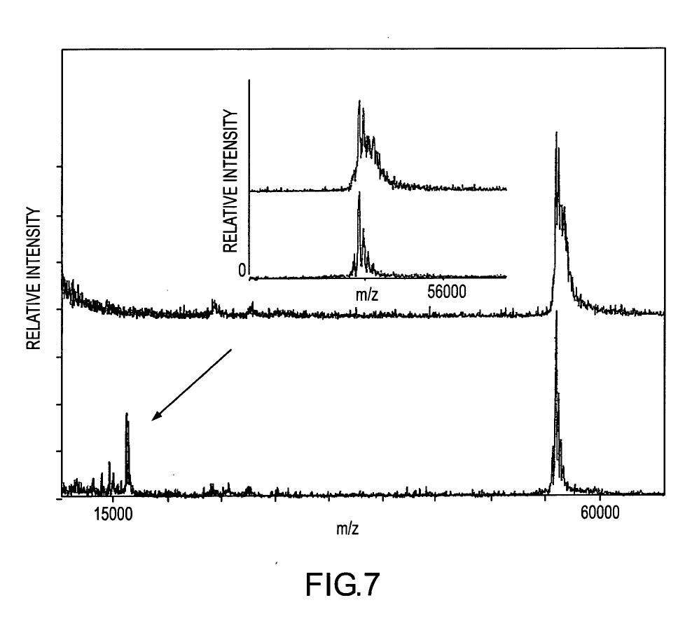

FIG. 7 shows MSIA spectra of VDB analyzed from human plasma. The protocols employed in the analysis were the same as those described for the plasma assay listed in Example 2. Analyses were performed on two individuals. Differences in glycosylation pattern are observed, as well as the presence of a truncated form in one individual.

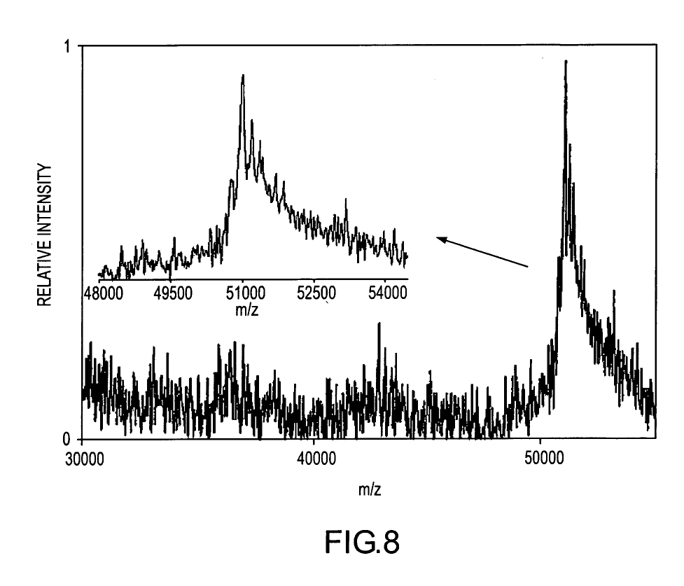

FIG. 8 shows MSIA spectra of VDB analyzed from urine. The protocols employed in the analysis were the same as those described for the urine assay listed in Example 2.

These VDB MSIA analyses of plasma and urine may be used to screen populations for genetic variants that may influence the transport of vitamin D as well as possibly assess the potential of an individual developing certain cancers.

### EXAMPLE 4

### UP1 MSIA

Urinary protein 1 (UP1, also known as Clara cell protein, CC10 or CC16) is an important peripheral biomarker for a variety of pulmonary ailments and urinary tract dysfunctions. UP1 is primarily secreted by Clara cells in the bronchioalveolar lining in mammalian lung tissue. Respiratory tract damage increases the plasma and urine levels of UP1 due to increased bronchoalveolar permeability and the overloading of the tubular reabsorption process, respectively. Furthermore, increased UP1 concentration in urine is an indication of proximal tubular dysfunction, whereas decreased UP1 plasma levels have been found in smokers, subjects suffering from asthma and schizophrenics. Normal concentrations of UP1 in plasma and urine are ˜15 μg/L and ˜3 μg/L, respectively.

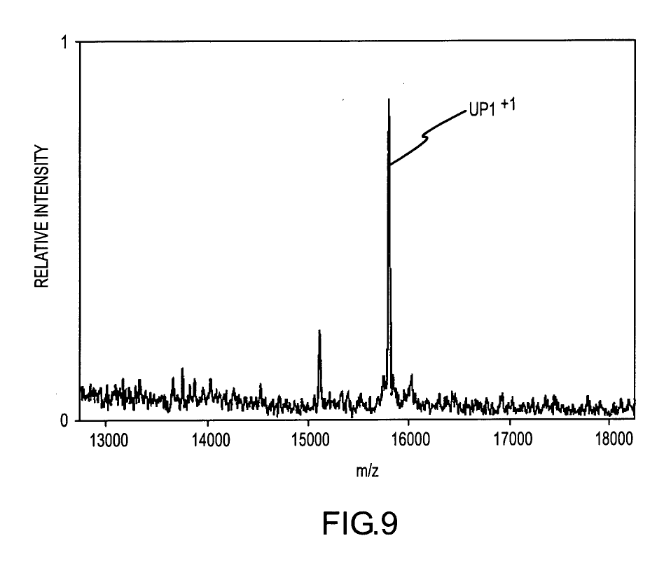

FIG. 9 shows MSIA spectra of UP1 analyzed from human plasma. The protocols employed in the analysis were the same as those described for the plasma assay listed in Example 2 but utilize a full 50 μL of plasma.

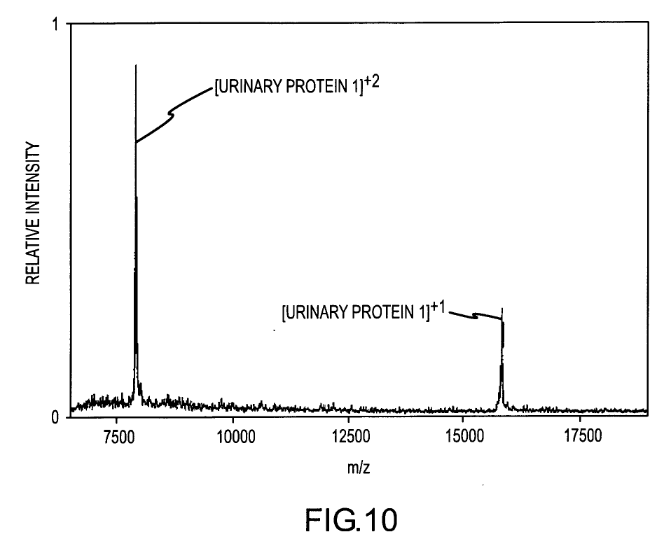

FIG. 10 shows MSIA spectra UP1 analyzed from urine. The protocols employed in the analysis were the same as those described for the urine assay listed in Example 2.

The population screening of plasma and urinary UP1 may be used to assess proximal tubual kidney function as well as monitor membrane permeability between the lung/blood barrier.

### EXAMPLE 5

### Bence Jones Proteins

The term Bence-Jones proteins generally refers to the free light chain monoclonal antibodies present in serum, urine or other body fluids. There are several types of BJP, most notably of the lambda (L) and the kappa (k) subtype. They exist either as monomers (at ˜22 kDa) or covalently/non-covalently-linked dimmers (˜at 44 kDa). BJP are by far the most important urinary monoclonal components, because of their clinical implications. Their presence in urine at high concentration is strongly indicative of malignant B-cell neoplasms. BJP are more easily detected in urine because they are filtered freely from the serum and into the urine.

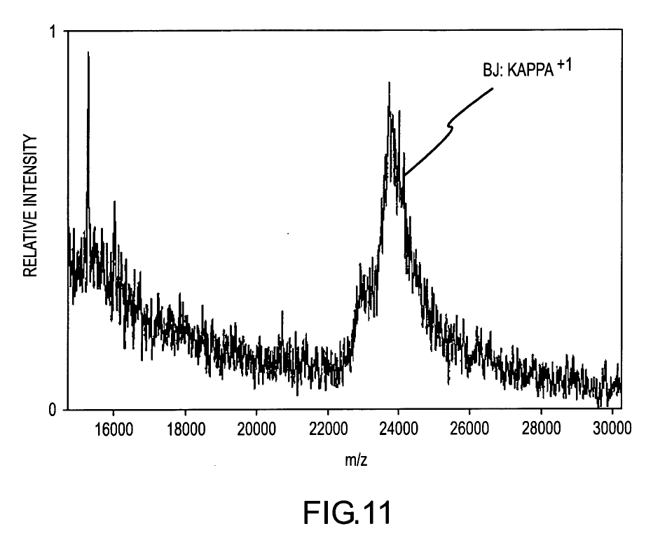

FIG. 11 shows MSIA spectra BJ-k analyzed from urine. The protocols employed in the analysis were the same as those described for the urine assay listed in Example 2.

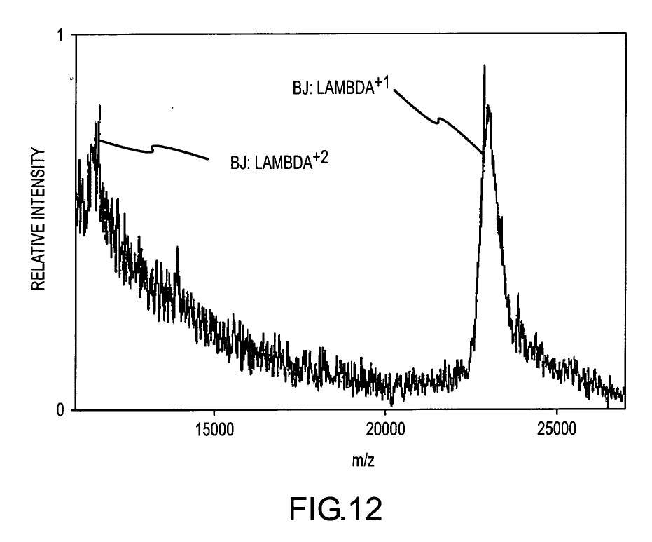

FIG. 12 shows MSIA spectra BJ-L analyzed from urine. The protocols employed in the analysis were the same as those described for the urine assay listed in Example 2.

The population screening of the BJ-lambda and kappa proteins may be used to assess individuals of the presence of certain cancers.

### EXAMPLE 6

### Insulin Like Growth Factor (Somatomedin C)

Insulin-like growth factor I (IGF-1), along with its homologue, IGF-2, are structurally and functionally related to insulin, but with a much higher growth-promoting activity. IGF-1 regulates cell activities involving cell proliferation, differentiation and apoptosis. More than 95% of IGF-1 circulates in plasma bound to IGF-binding proteins (IGFBPs 1-6), although the free form (7.5 kDa) is considered to be the active form (in the same way as insulin). The circulating levels of IGF-1 vary throughput life, increasing from birth to puberty and decreasing steadily after the third decade. Recent demographic study of the IGF-1 levels found comparable values (˜150 ng/mL) in both white and African American men. Currently, standard radioimmunoassays are used for IGF-1 measurement in plasma. Although conflicting reports exist, more and more studies indicate the correlation between increased plasma levels of IGF-1 and the development of prostate cancer. It has been shown that IGF-1 is required for the normal development and growth of the prostate gland.

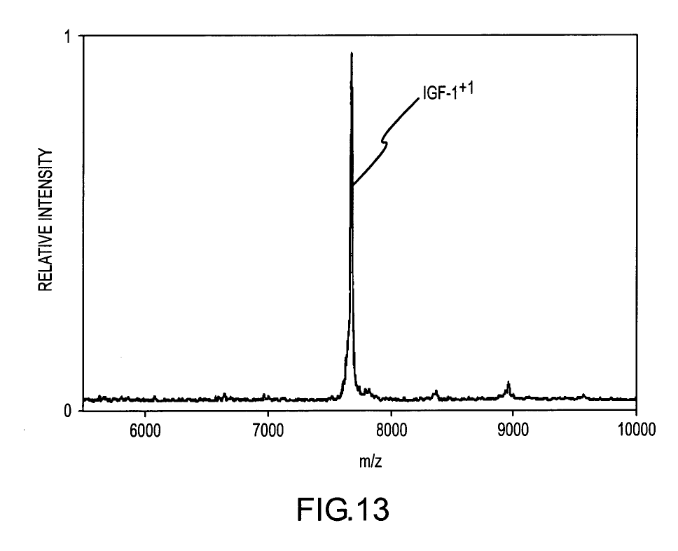

FIG. 13 shows MSIA spectra of IGF1 analyzed from human plasma. The protocols employed in the analysis were the same as those described for the plasma assay listed in Example 2 but uses a full 50 μL of plasma that is pretreated 1:1 with 0.5% sodium dodecy sulphate (SDS) and then diluted to 1 mL with HBS. Also, α-cyano-4-hydroxycinnamic acid was used as the MALDI matrix.

### EXAMPLE 7

### Serum Amyloid A

Serum amyloid A (SAA) is an apolipoprotein of the HDL particles with a MW=11,682. SAA is a polymorphic protein, consisting of several genetic isotypes (three of which are present in human plasma). SAA plasma concentration ranges from 1 to 1000 mg/L, depending on the inflammatory conditions. Although SM has been suggested as possible inflammation biomarker, the difficulty associated with its purification and assaying directly from plasma has prevented its wider use in clinical studies. SAA residues 50-76 form insoluble fibrils in extracellular spaces, leading to a disorder called reactive amyloidosis (seen in rheumatoid arthritis and tuberculosis).

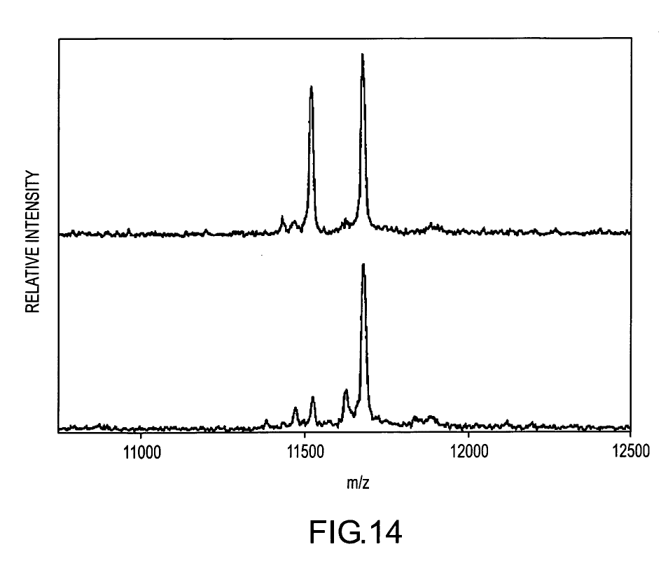

FIG. 14 shows MSIA spectra of SM analyzed from human plasma. The protocols employed in the analysis were the same as those described for the plasma assay listed in Example 2, but use 50 μL of plasma. Analyses were performed on two individuals from which differences in the amount of truncated SAA are observed.

The population screening of SAA may be used to determine the presence of certain inflammatory disorders as well as evaluate an individual's susceptibility to certain amyloid related syndromes.

### EXAMPLE 8

### Leptin

Leptin (LEP) is an adipocyte protein hormone that functions as an afferent signal in a negative feedback loop regulating body weight. In lean persons with minimal adipose tissue, the majority of leptin circulates bound to other proteins (such as the soluble leptin receptor). In obese people, the majority of leptin circulate as free (unbound) leptin. The molecular weight of leptin is 16,026, and its concentration ranges from ˜5 μg/mL for the bound from, to ˜10 and ˜30 mg/mL for the free form in lean and obese subjects, respectively.

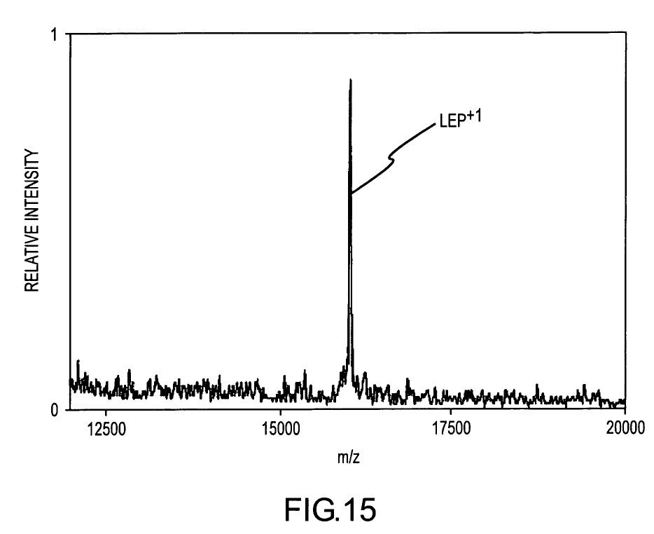

FIG. 15 shows MSIA spectra of LEP analyzed from human plasma. The protocols employed in the analysis were the same as those described for the plasma assay listed in Example 2, but utilize 100 μL of plasma diluted 1:4 with HBS buffer.

### EXAMPLE 9

### Tamm-Horsfall Protein

Tamm-Horsfall glycoprotein (THG, also known as Uromodulin) is a glycoprotein produced in the kidney by the cells of the ascending loop of Henle and adjacent convoluted tubule. THG is the most abundant protein present in urine of healthy people. It is a ˜85 kDa glycoprotein (639 amino acids backbone), and it contains eight potential glycosylation sites (at least five of which are occupied by complex sugar chains). The biological role of THG in kidney, although extensively studied, remains unclear. It has been suggested that the N-glycans of THG are involved in the prevention of urinary tract infections, and in the immunosuppressive function. Some studies demonstrated its role in the regulation of circulating levels and biological activity of certain cytokines. And, finally, it may play a role in renal stone formation, and may be involved in the process of urine dilution and concentration.

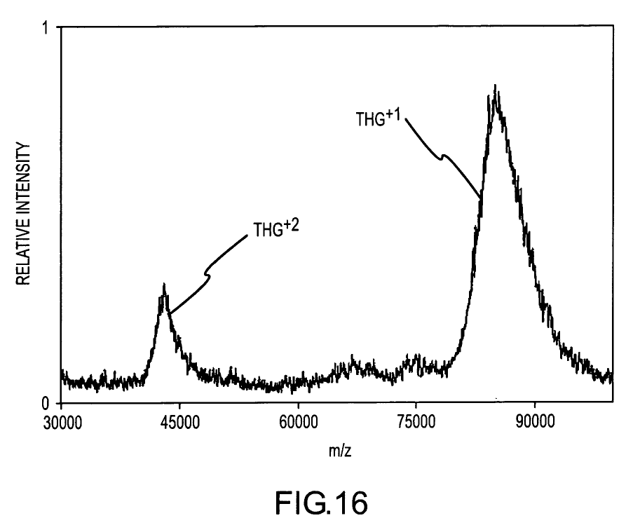

FIG. 16 shows MSIA spectra of THG analyzed from urine. The protocols employed in the analysis were the same as those described for the urine assay listed in Example 2.

### EXAMPLE 10

### Albumin

Albumin (ALB) is a soluble monomeric protein which comprises about half of the blood serum proteins. This 65 kDa protein serves primarily as a carrier of steroids, fatty acids, and thyroid hormones as well as a stabilizer of extracellular fluid volume. ALB is also found in urine at a concentration of 30 mg/L.

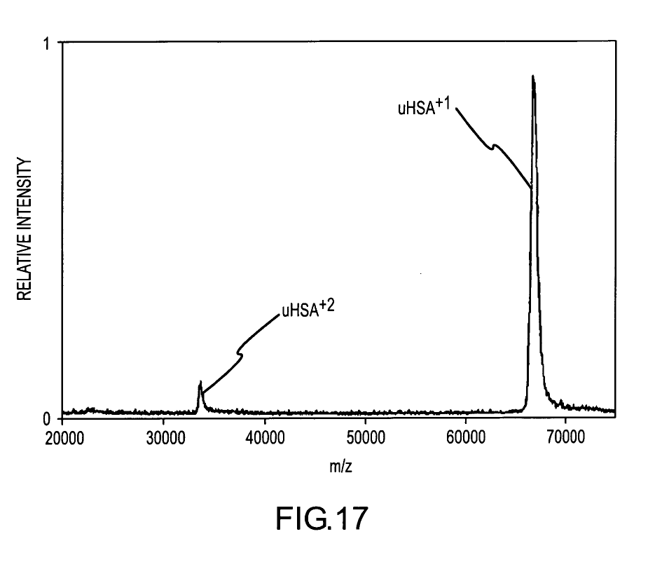

FIG. 17 shows MSIA spectra of ALB analyzed from urine. The protocols employed in the analysis were the same as those described for the urine assay listed in Example 2. Analyses were performed on two individuals showing great variation in the broadness of the ALB signals, suggesting the detection of a modified form of ALB either by lipid association or pharmacological modification.

### EXAMPLE 11

### Lysozyme C

Lysozyme C (LYC also known as muramidase) functions as an antimicrobial enzyme by hydrolyzing the bacterial cell wall beta (1-4) glycosidic linkages between N-acetylmuramic acid and N-acetylglucosamine. With a molecular mass of 14,062 Da, lysozyme is found in variety of tissues and biological fluids, including plasma and urine.

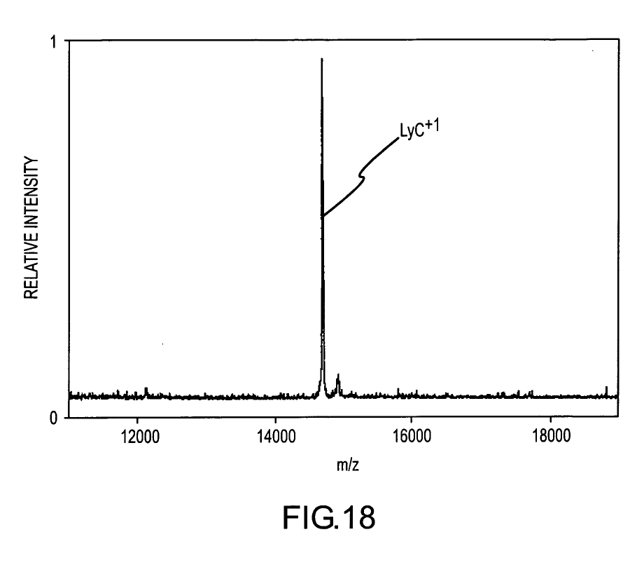

FIG. 18 shows MSIA spectra of LYC analyzed from human plasma. The protocols employed in the analysis were the same as those described for the plasma assay listed in Example 2.

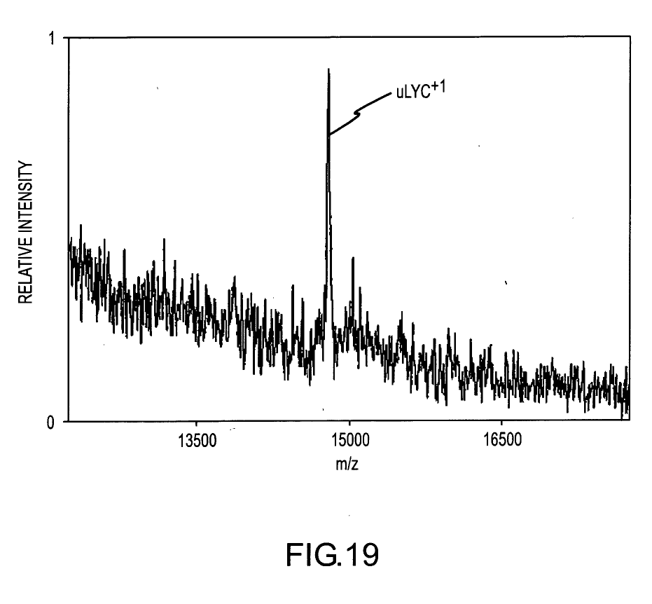

FIG. 19 shows MSIA spectra of LYC analyzed from urine. The protocols employed in the analysis were the same as those described for the urine assay listed in Example 2.

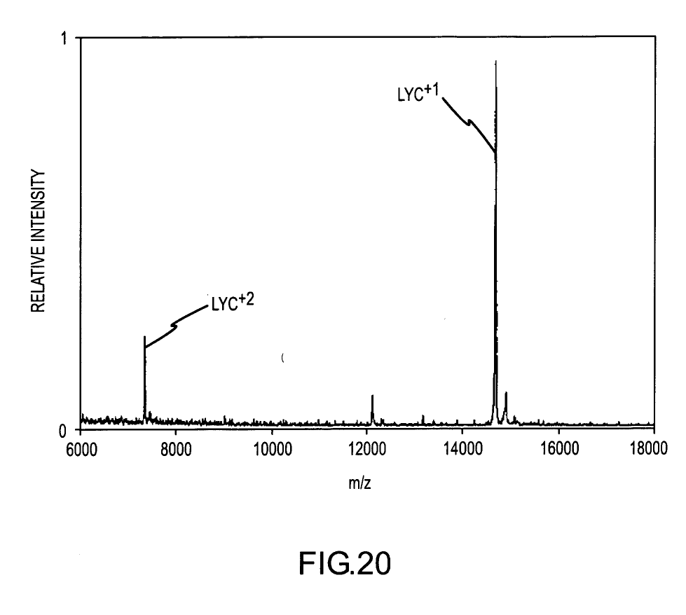

FIG. 20 shows MSIA spectra of LYC analyzed from saliva. The protocols employed in the analysis were the same as those described for the saliva assay listed in Example 2.

The population screening for LYC may be used to identify the presence of genetic variants that are associated with certain amyloid disorders.

### EXAMPLE 12

### A-Defensins

The α-defensins, also known as human neutrophil defensins (HNP), are a family of cysteine-rich, cationic antimicrobial peptides secreted from neutrophils. HNP are readily found in multiple biological fluids including plasma, urine, saliva and sputum and are believed to increase in concentration with the presence of certain malignancy and bacterial infections.

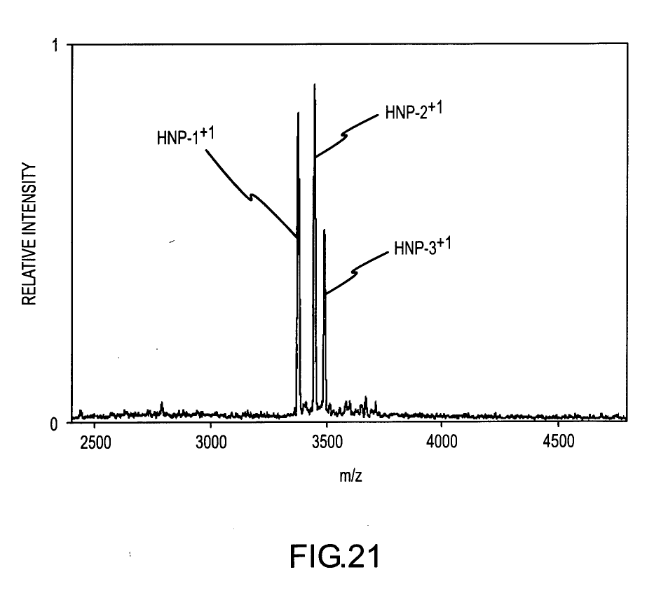

FIG. 21 shows MSIA spectra of HNP analyzed from urine. The protocols employed in the analysis were the same as those described for the urine assay listed in Example 2, but used α-cyano-4-hydroxycinnamic acid as the MALDI matrix.

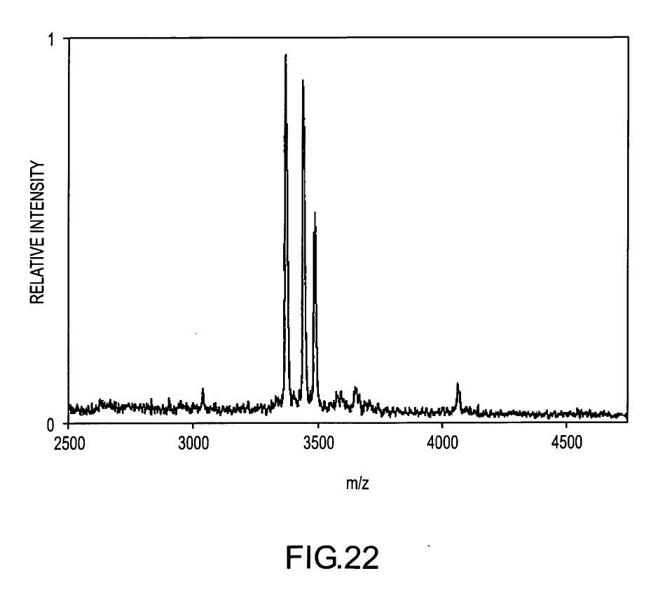

FIG. 22 shows MSIA spectra of HNP analyzed from saliva. The protocols employed in the analysis were the same as those described for the saliva assay listed in Example 2, but used α-cyano-4-hydroxycinnamic acid as the MALDI matrix.

### EXAMPLE 13

### Immunoglobulin G

Immunoglobulin G (IgG) is one of the five classes of a group of proteins called immunoglobulins. IgG has a strong affinity to protein A and is routinely purified using Protein A columns. As a part of the immune response, immunoglobulins, including IgG, consist of four subunits: two identical light and heavy chains, held together by disulfide and non-covalent interactions to form a Y-shaped symmetric dimer. The role of these glycosylated proteins is the recognition of a specific bio-molecular target, or antigen, for subsequent destruction by the host immune system. IgG is readily found in both plasma and urine of humans.

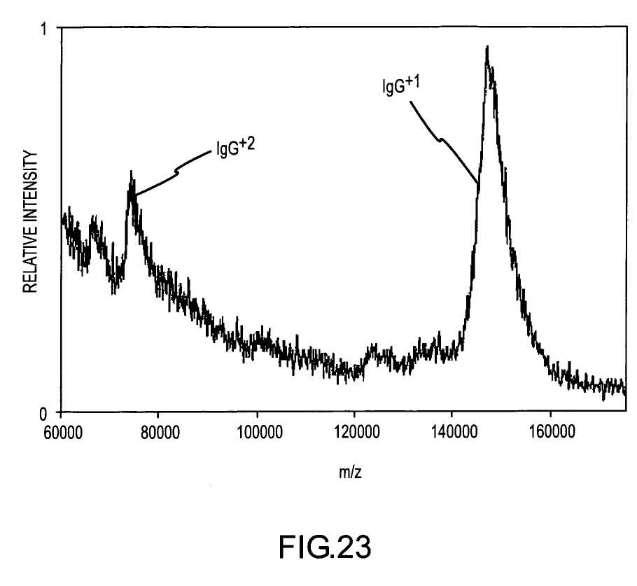

FIG. 23 shows MSIA spectra of IgG analyzed from urine. The protocols employed in the analysis were the same as those described for the urine assay listed in Example 2, but with Protein A MSIA Tips that were prepared in the same protocols described above.

### EXAMPLE 14

### Serum Amyloid P Component

Serum amyloid P component (SAP) is a high plasma level (mid mg/L range) glycoprotein found in humans and many other species of animals. As a member of the pentaxin family, SAP has been found to be a minor acute phase response marker, but its primary purpose is still largely unknown. With a 203 amino acid sequence, the entire SAP homopentamer complex is over 225 kDa. Serum amyloid P has been widely identified associated with rogue DNA, histones and amyloid fibrils in human plasma, acting as a shield from autoimmune response. High concentrations of serum amyloid P and component-P deposits have been associated with Alzheimer's and Family Amyloid Polyneuropathy plaques while some research suggests that SAP complexation to amyloid fibrils may deter β-amyloid plaque formation.

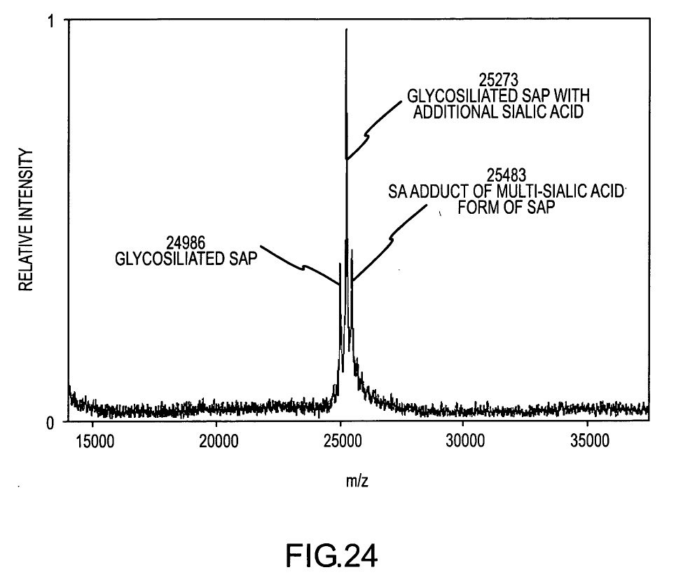

FIG. 24 shows MSIA spectra of SAP analyzed from plasma. The protocols employed in the analysis were the same as those described for the urine assay listed in Example 2.

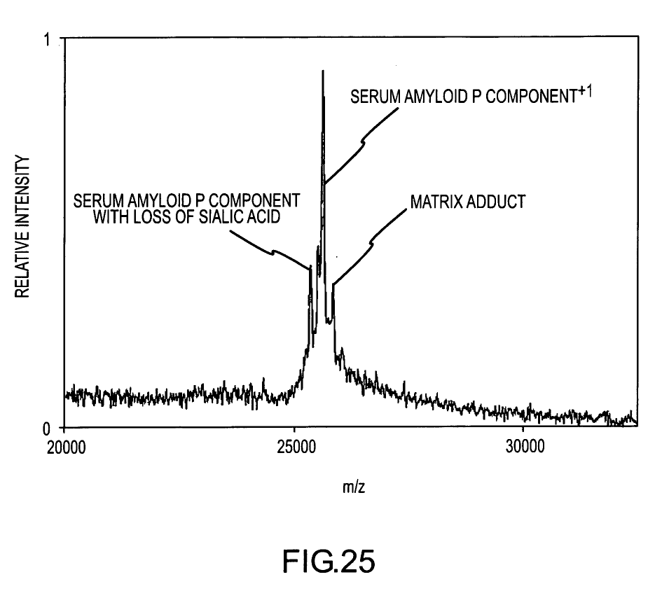

FIG. 25 shows MSIA spectra of SAP analyzed from urine. The protocols employed in the analysis were the same as those described for the urine assay listed in Example 2.

### EXAMPLE 15

### Retinol Binding Protein

Retinol binding protein (RBP) is a member of the lipocalin family of proteins. All proteins in this family are characterized by an ability to bind small, primarily hydrophobic molecules for transport throughout the body. The primary ligand for RBP is all-trans retinol (vitamin A). The lipocalins are also known to form complexes with other proteins. At 21,065 Da, RBP would normally be filtered from plasma under normal glomerular function because of its small size. In its holo-form (bound to retinol), RBP becomes complexed to the transthyretin (TTR) tetramer (54 KDa), allowing for the retinol-containing complex to be retained in the plasma. Loss of the C-terminal leucine targets RBP for glomerular filtration because the truncated RBP cannot associate with TTR. Persons suffering from chronic renal failure have accumulating amounts of RBP in their plasma. Normal retinol binding protein plasma levels are in the 50 mg/L range while low RBP plasma concentrations are associated with vitamin A deficiency.

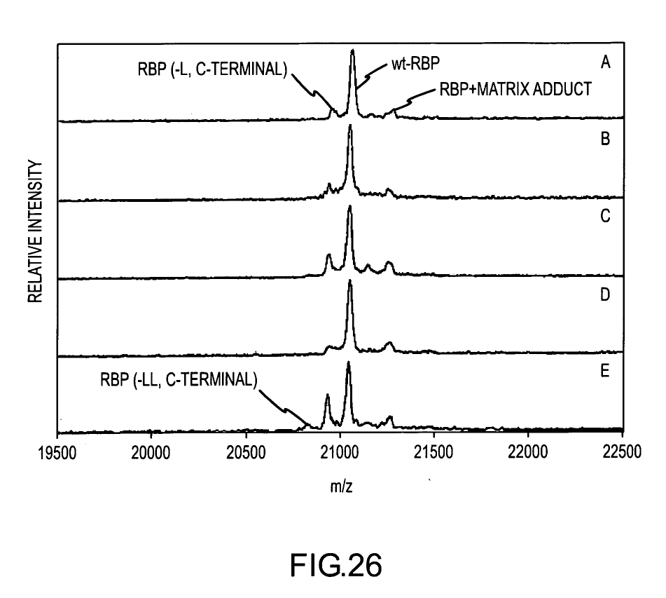

FIG. 26 shows MSIA spectra of RBP analyzed from human plasma. The protocols employed in the analysis were the same as those described for the plasma assay listed in Example 2. Analyses were performed on five individuals, four healthy controls and one renal failure patient. Differences in the amount of truncated RBP vary greatly between individual with healthy kidneys and those with renal impairment resulting in differentiable RBP patterns.

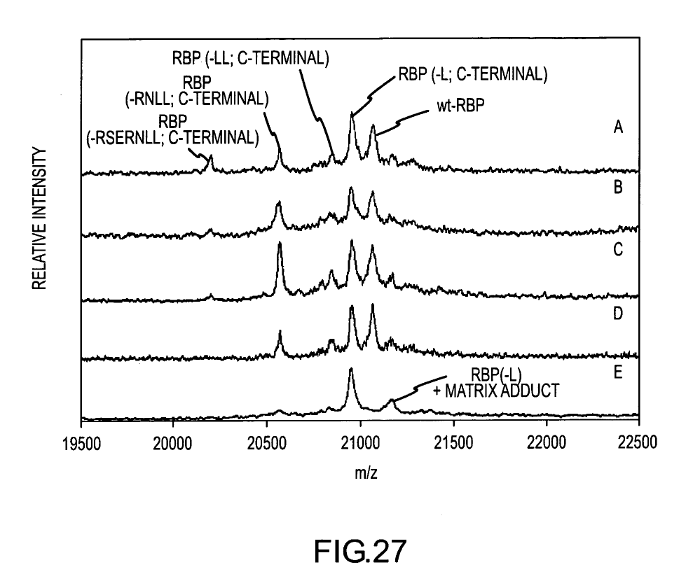

FIG. 27 shows MSIA spectra of RBP analyzed from urine. The protocols employed in the analysis were the same as those described for the urine assay listed in Example 2. Analyses were performed on the same five individuals above. Observable differences in the protein pattern of the RBP variants are shown between the healthy controls and the diseased individual.

Population screening of RBP may be used to identify genetic variants of RBP as well as assess the kidney function of individuals.

### EXAMPLE 16

### C-Reactive Protein

A pentaxin protein, C-reactive protein (CRP) is a clinical marker for acute phase response to inflammation. Monomeric CRP (23,045 Da) forms a homopentamer complex (over 200 kDa) having a calcium dependent affinity for phosphocholine within c-polysaccharide present in the cell wall. C-reactive protein has also been shown to facilitate phagocytosis, aiding innate immunity and opsonization. Studies have shown CRP levels can increase 1000 fold in response to rheumatoid arthritis, bacterial infection and coronary malfunction. Typical clinical assays set a plasma level of CRP>1-2 mg/L as an indicative threshold for possible disease state or infection, but there is a lot of variation in the literature as to what the basal levels of CRP are. C-reactive protein is often screened in tandem with serum amyloid A (SAA) and/or procalcitonin for potential acute disease or infectious states.

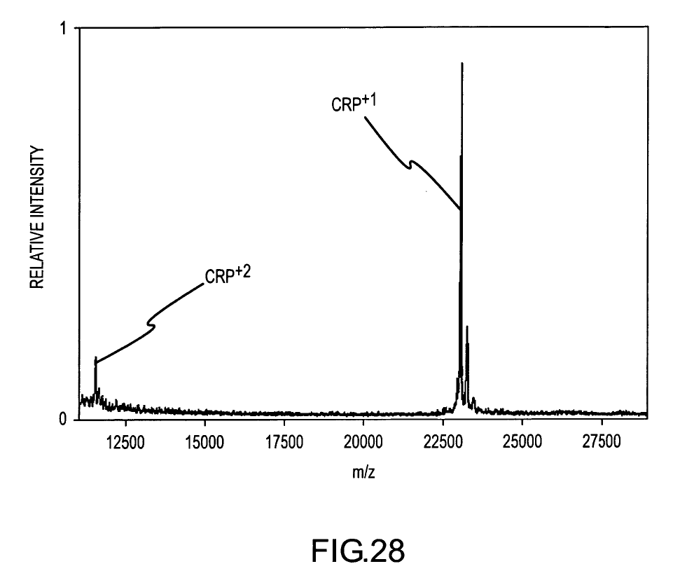

FIG. 28 shows MSIA spectra of CRP analyzed from human plasma. The protocols employed in the analysis were the same as those described for the plasma assay listed in Example 2, but requires 50 μL of plasma that is pretreated with EDTA.

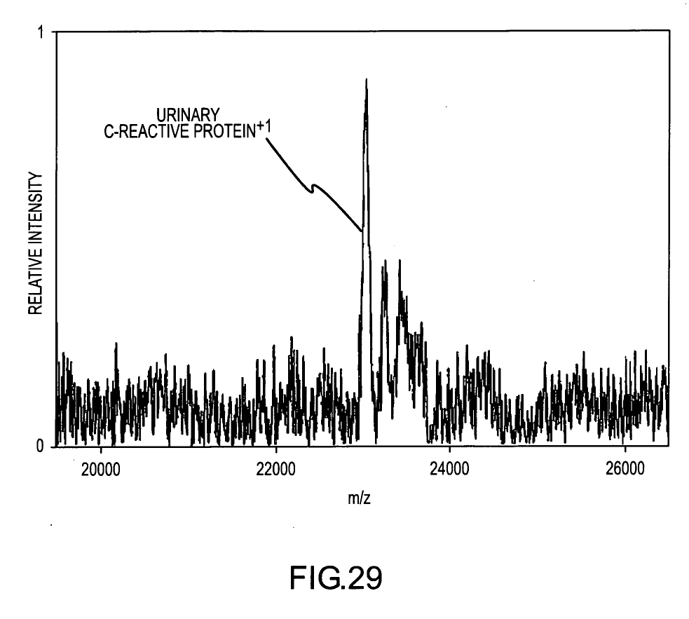

FIG. 29 shows MSIA spectra of CRP analyzed from urine. The protocols employed in the analysis were the same as those described for the urine assay listed in Example 2.

### EXAMPLE

### 17

### Multi-Analyte RBP/CRP/SAP—A Standard Addition

The use of MSIA is not limited to single protein analyses. Multiple affinity ligands may be coupled to the same MSIA-Tip. The procedure for producing such multi-analysis MSIA-Tips is unaltered from the previously mentioned method except uses an IgG cocktail towards all protein species to be simultaneously targeted. The combination of affinity ligands towards RBP, CRP and SAP allows for the generation of a protein profile of these three proteins, and their variants, from which protein ratios using peak intensity/integral may be generated. Absolute quantitation is readily achievable with this method by use of standard addition. Serial additions of highly purified standard CRP solution to plasma samples allows for the generation of a calibration curve relating the relative peak intensities with a CRP concentration value.

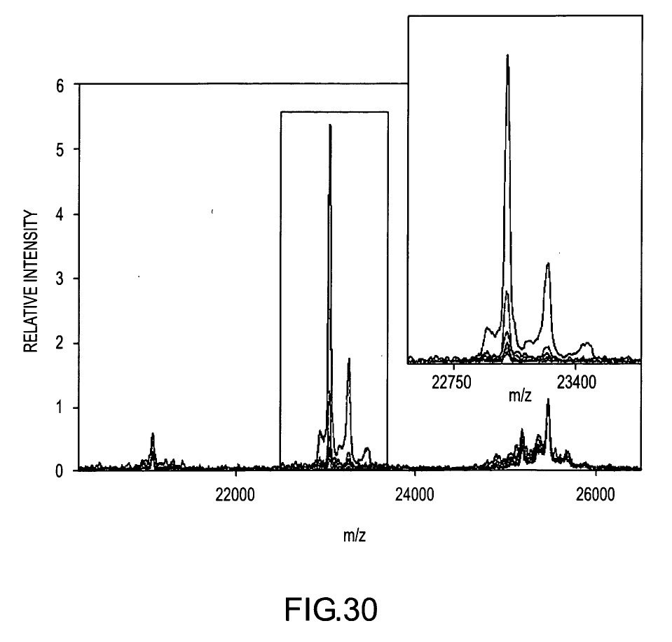

FIG. 30 shows the RBP/CRP/SAP multi-analyte MSIA spectra of multiple plasma samples undergone CRP standard addition. Sample interrogation was the same as described in Example 2, but used 50 μL of plasma per sample which was pre-treated with EDTA and subject to the addition of CRP standard. Signals from RBP, CRP and SAP are present, but samples with increased amounts of standard CRP have a greater CRP signal intensity.

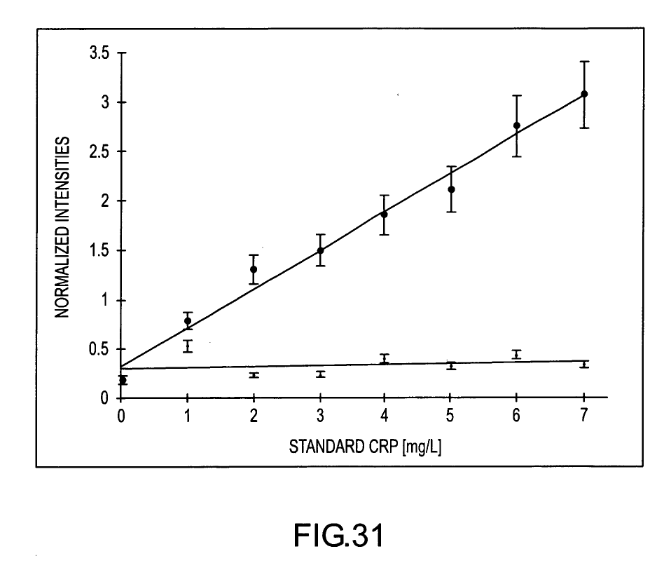

FIG. 31 shows the resulting calibration curve generated from the CRP standard addition. Both the intensity relationships of RBP/SAP and CRP/SAP towards the CRP standard addition are shown. No change in the RBP intensity is seen with the addition of CRP standard, while CRP is incrementally increasing. The standard curve of normalized CRP intensities, with an average error of 11.97%. Linear regression of the plot determined the native C-reactive protein concentration to be 0.8110 mg/L.

### EXAMPLE 18

### High Throughput Population Profiling—RBP/CRP/SAP

The application of multi-analyte MSIA towards population profiling does not require absolute quantitation to identify differences in human protein expression levels. Instead, relative quantitative comparisons between RBP, CRP and SAP are rapidly capable of determining if an individual has significantly different protein levels from the rest of the population.

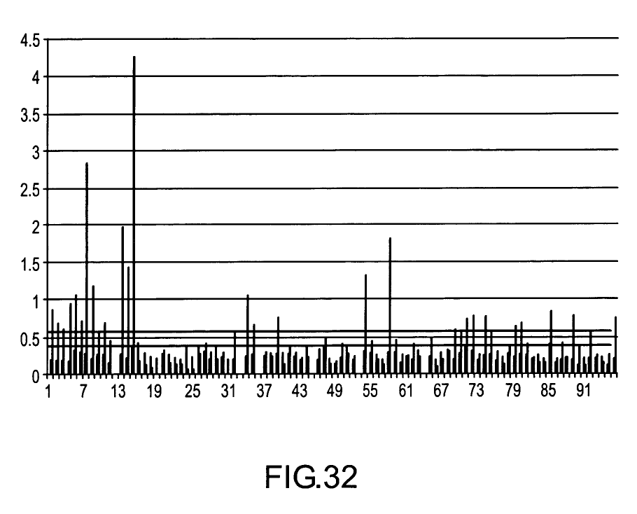

FIG. 32 is a histogram illustrating how differences in relative plasma CRP levels 96 individuals using high throughput multi-analyte MSIA can be readily displayed. Individuals with higher levels of CRP in their plasma have higher amplitude CRP/SAP protein ratios, as shown in FIG. 32. The protocols employed in the analysis were the same as those described in Example 2, but used 50 μL of plasma pretreated with EDTA.

### EXAMPLE

### 19

### Transthyretin

Transthyretin (TTR) is a small protein produced in the liver and found in serum and cerebral spinal fluid as a homotetramer. Functionally, TTR serves unaccompanied in the transport of thyroid hormones, or in complexes with other proteins in the transport of various biologically active compounds. Structurally, wild-type (wt) TTR is comprised of 127 amino acids and has a molecular weight (MW) of 13,762.4. Over eighty point mutations have been cataloged for TTR, with all but ten potentially leading to severe neurological complications. The majority of mutation-related disorders are caused by amyloid plaques depositing on neurons or tissue, eventually leading to dysfunctions including carpal tunnel syndrome and familial amyloid polyneuropathy.

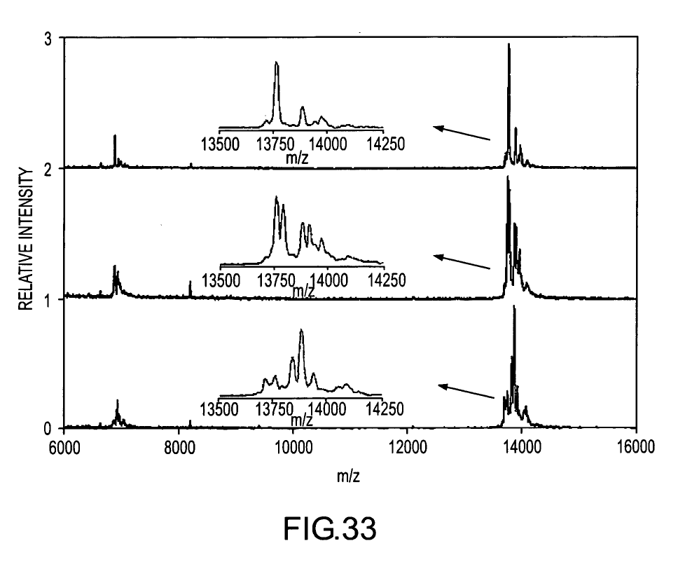

FIG. 33 shows MSIA spectra of TTR analyzed from human plasma. The protocols employed in the analysis were the same as those described for the plasma assay listed in Example 2. Analyses were performed on three individuals. The presence of a point mutation in one individual is readily apparent while the other two samples show differences in the degree of posttranslational modifications.

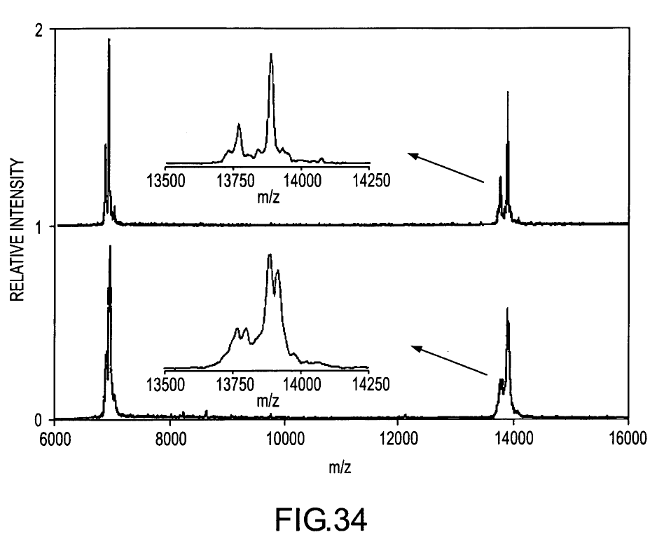

FIG. 34 shows MSIA spectra of TTR analyzed from urine. The protocols employed in the analysis were the same as those described for the urine assay listed in Example 2. Analyses were performed on the same three individuals. Again, the presence of a point mutation in one sample is readily apparent while the others show variation in the degree of PTM.

The population screening of TTR may be used to identify genetic variants, which may lead to amyloid disorders, as well as identify aberrant PTMs that may lead to dysfunctional forms of the protein associated with certain endocrine disorders.

### EXAMPLE 20

### Transferrin

Transferrin (TRFE) is the major iron transport protein found in human plasma with basal levels in the mid g/L range. This large monomeric glycoprotein (79.6 kDa) consists of 679 amino acids and has two sites for asparagines-linked glycosylation. There are seven differently branching glycosylated forms studied for the identification of carbohydrate deficient glycoprotein syndrome (CDGS) and chronic alcohol abuse. Due to its high variability of glycosilation levels (from normal to diseased state) and its high concentration in plasma, TRFE has become the standard method of monitoring CDGS and its follow up treatment. Current clinical method of monitoring TRFE is through isoelectric focusing gels.

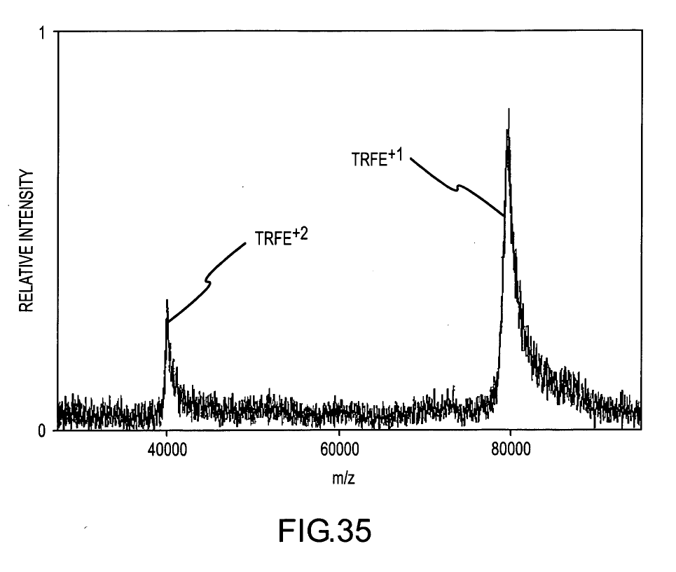

FIG. 35 shows MSIA spectra of TRFE analyzed from human plasma. The protocols employed in the analysis were the same as those described for the plasma assay listed in Example 2.

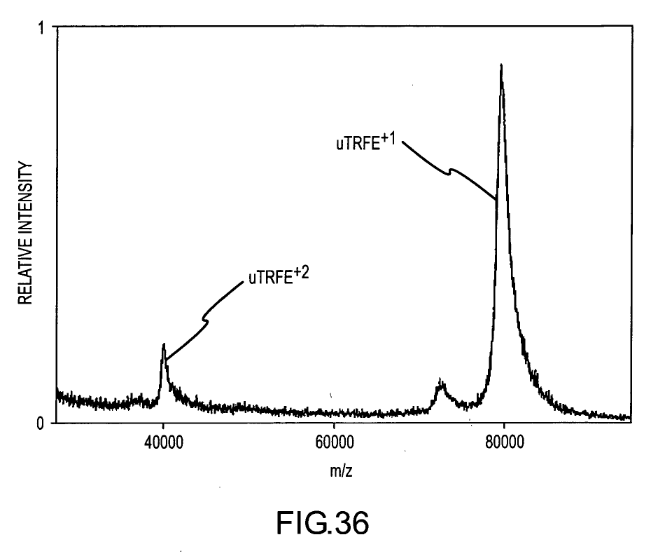

FIG. 36 shows MSIA spectra of TRFE analyzed from urine. The protocols employed in the analysis were the same as those described for the urine assay listed in Example 2.

Population screening of TRFE may be used to assess glomerular filtration of individuals as well as identify variant forms of TRFE that may be associated with alcoholism and/or carbohydrate deficient glycoprotein syndrome I and/or II.

### EXAMPLE 21

### Apolipoprotein E

Apolipoprotein E (apoE) is a 34 kDa protein that associates with both the high density lipoprotein (HDL) and very low density lipoproteins (VLDL). Apo E has three major isoproteins, E2, E3 and E4, with E3 being the most common. Individuals that express Alzheimer's disease have been found to have the apo E4 allele in their phenotype. On the other hand, those individuals whose phenotypes include the apo E2 allele have shown increased risk for type III HLP (hyperlipoproteinemia).

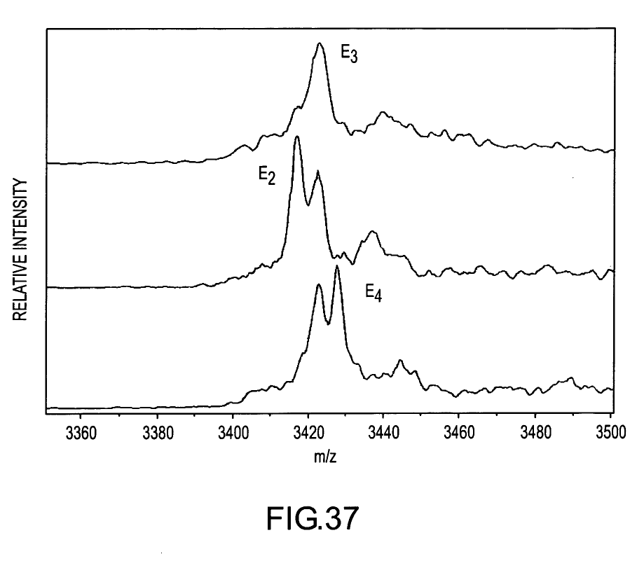

FIG. 37 shows MSIA spectra of ApoE analyzed from human plasma. The protocols employed in the analysis were the same as those described for the plasma assay listed in Example 2, but uses 50 μL of plasma pretreated with 20 μL of 1% Tween-20. Analyses were performed on three individuals. The presence of point mutations of in two of the samples is observed which are identified as the ApoE3 and ApoE4 phenotypes.

The population screening of ApoE may be used to identify genetic variants of the protein within individuals that may be associated several disorders including alzheimer's disease.

### EXAMPLE 22

### Apolipoprotein AI

The function of apolipoprotein A-I (apo A-I), as is the function of all apolipoproteins, is to stabilize lipids during their transportation through the circulatory system. Typically, 90% of all plasma apo A-I is coupled with high density lipoproteins (HDL). These lipoproteins are implicated in a state of elevated cholesterol associated with lowered risk of artherosclerosis. Monitoring levels of plasma apolipoprotein A-I constitutes a potential biomarker for determining this degree of patient risk. Hence, an assay is needed to establish the quantity and quality, i.e post-translational modifications, of apo A-I for use as a biomarker in determining the degree of risk for atherosclerosis and related diseases.

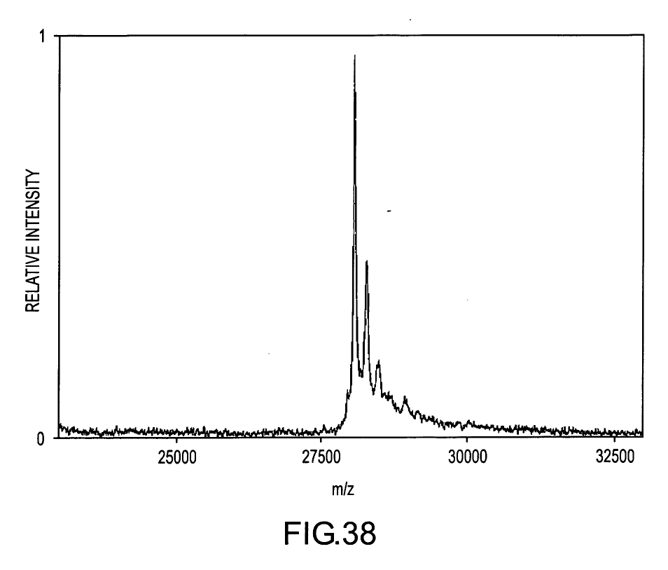

FIG. 38 shows MSIA spectrum of ApoA-1 analyzed from human plasma. The protocols employed in the analysis were the same as those described for the plasma assay listed in Example 2, but use samples pretreated with 1% Tween-20.

### EXAMPLE 23

### Apolipoprotein AII

Apolipoprotein A-II (ApoA-II) is a member of the apolipoprotein family. In plasma, it is associated with high-density lipoprotein (HDL). It exists both as a monomer (MW=8,707) and as a disulfide-linked homodimer (MW=17,414). ApoA-II has been found to form a complex with apolipoprotein E (ApoE), which gives rise to the association of ApoA-II with the pathogenesis of Alzheimer's disease (AD) through the reduction of intracellular β-amyloid (Aβ). The effects of ApoA-II-mediated binding of ApoA-I to the HDL particles are also a subject of interest. In all, the role of ApoA-II is just being discovered in a number of important biological processes.

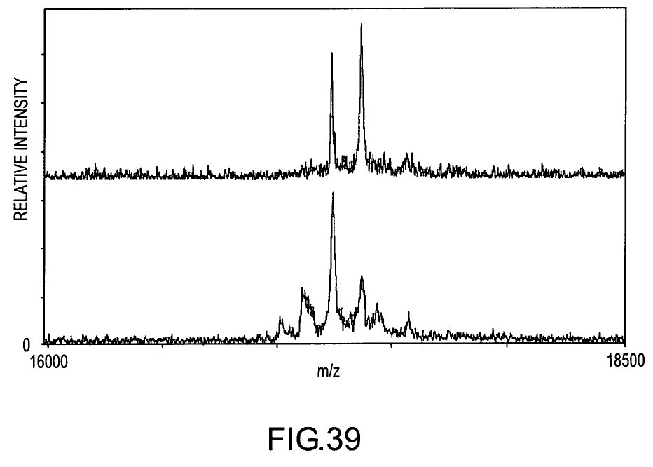

FIG. 39 shows MSIA spectra of ApoA-II analyzed from human plasma. The protocols employed in the analysis were the same as those described for the plasma assay listed in Example 2, but samples were pretreated with 1% Tween-20. Analyses were performed on two individuals. Signals observed were from the ApoA-II homodimer with noticeable differences in truncation between the two samples.

### EXAMPLE 24

### Biotinylated Polypeptides

The vitamin Biotin and the protein Avidin form one of the strongest non-covalent bonds between biological molecules. As such, their interaction is oftentimes exploited for recognition events of other biomolecules. In most instances, avidin derivatized solid supports are used for affinity retrieval of biotin labeled (biotinylated) biomolecules. Avidin MSIA-Tips were prepared using the same protocols for IgG immobilization.

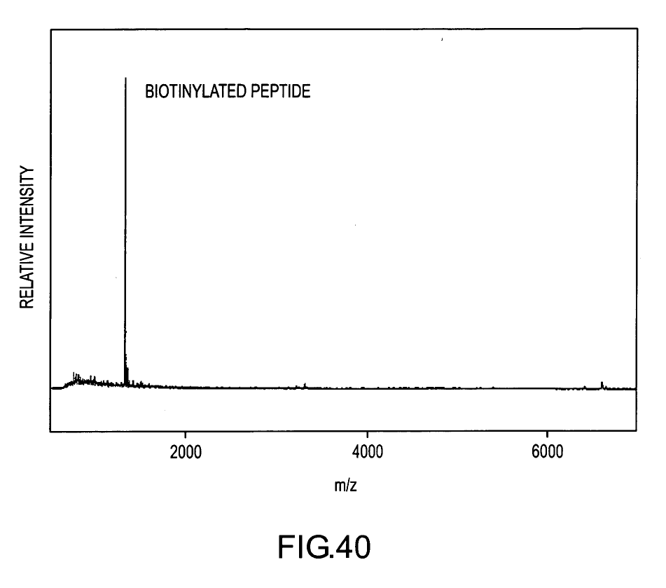

FIG. 40 shows the MSIA analysis of a biotinylated peptide from human plasma. The protocols employed in the analysis were the same as those described for the plasma assay listed in Example 2, but samples were spiked with a 1 μM solution of a biotin-labeled peptide (MW=1,338.40) and α-cyano-4-hydroxycinnamic acid was used.

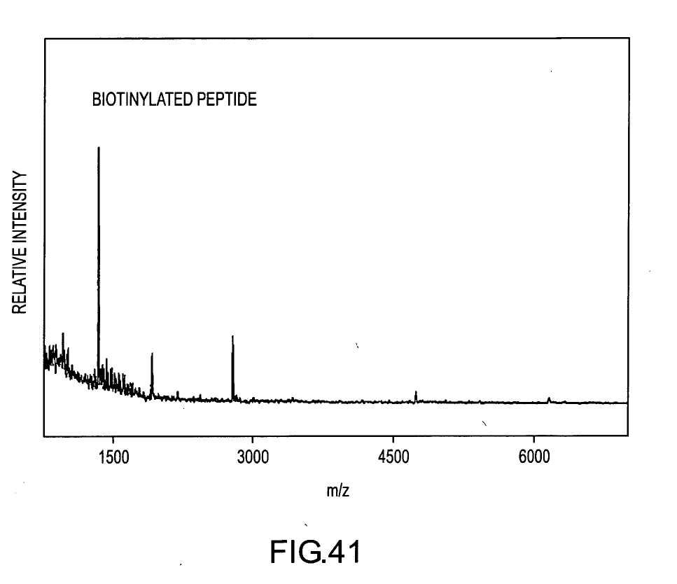

FIG. 41 shows a MSIA spectrum of a biotinylated peptide analyzed from human urine. The protocols employed in the analysis were the same as those described for the urine assay listed in Example 2, but samples were spiked with a 1 μM solution of the same biotin-labeled peptide used above and α-cyano-4-hydroxycinnamic acid was used.

### EXAMPLE 26

### 6× His-Tagged Proteins

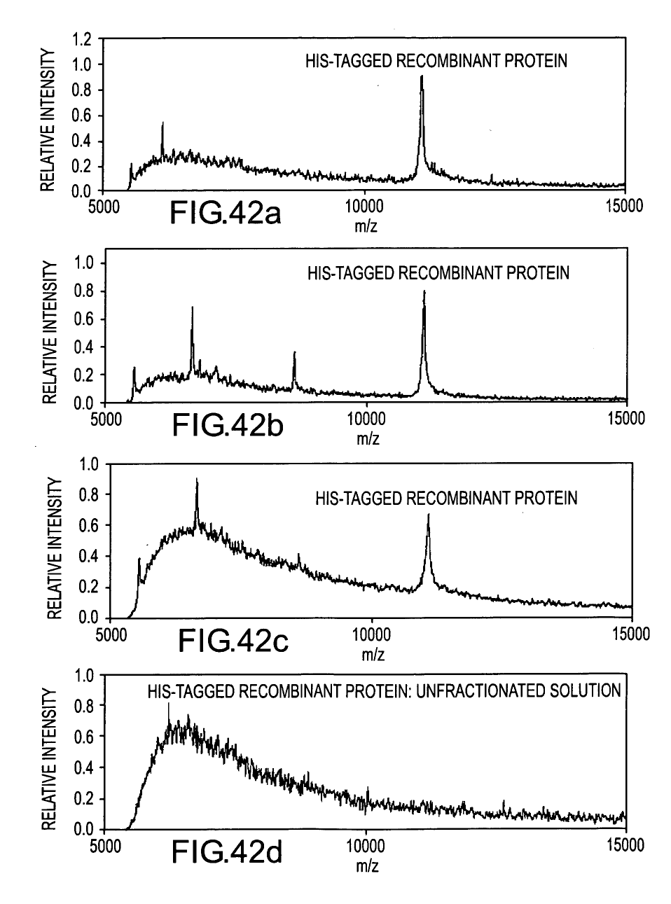

MSIA can be used in the analysis of a His-tagged recombinant protein from E. coli lysate. E. coli cells, expressing a recombinant His tag protein, were grown and harvested using standard procedures. Several milligrams of the cell pellet were resuspended in 200 μL of B-PER II Bacterial Protein Extraction Reagent (Pierce, Rockford, Ill. USA). The mixture was thoroughly agitated, vortexed and sonicated. Cell debris was removed by centrifugation at 13,000 RPM (9,000×g) for 5 minutes. The supernatant was decanted and mixed with 200 μL of a 0.5% (v/v) SDS solution. The solution was thoroughly agitated, vortexed and sonicated, and placed in a hot water bath (100° C.) for 5 minutes to enhance the solubilization of the proteins. To the 400 μL of this mixture was added a 400 μL HBS buffer. This solution (˜800 μL) was used as a sample for MSIA. Polyclonal anti-His MSIA-Tips were made in the same fashion as previously described. Chelating MSIA-Tips were made via NTA functionalized/CMD modified and CDI activated MSIA-Tips. The MSIA-Tips were used to rapidly extract targeted 6×His-tagged proteins expressed from cell culture. The sample solution was repetitively (50 times) aspired and dispensed (200 μL each time) through the anti-His and NTA-MSIA-Tips. A rinse with HBS (without the EDTA) (10 aspirations and dispensing, 200 μL each) and water (10×200 μL) followed. The captured protein was eluted from the MSIA-Tips with a small volume of MALDI matrix (saturated aqueous solution of α-cyano-4-hydroxycinnamic acid (ACCA), in 33% (v/v) acetonitrile, 10% (v/v) acetone, 0.4% (v/v) trifluoroacetic acid) and stamped onto a MALDI target array surface comprised of self-assembled monolayers chemically masked to make hydrophilic/hydrophobic contrast target arrays. The sample spot on the target array was analyzed using MALDI-TOF mass spectrometry. The resulting mass spectra are shown in FIGS. 42a-42d. FIG. 42a displays the result of the MSIA analysis utilizing the anti-His MSIA-Tip extraction of the His-tagged recombinant protein, demonstrating poignant reference point imparted to MSIA-NTA Tip. A major signal due to the singly charged ion of the His-tagged protein is observed. FIGS. 42b and 42c show mass spectra of the same His-tagged recombinant protein solution after processing with MSIA-NTA Tip, both in the presence and absence of nickel, respectively. It can be seen from FIGS. 42b and 42c that the major signal due to the singly charged ion of the His-tagged protein is enhanced in the presence of nickel. FIG. 42d shows mass spectrum of diluted nascent His-tagged recombinant protein solution. Signal from the His-tagged protein is not observed.

The present invention and the results shown in FIGS. 2 through 42 clearly demonstrate the usefulness of MSIA in the analysis of specific proteins and variants present in various biological fluids as well as the need for MSIA kits to expedite and enable the use of MSIA in analysis for specific proteins and variants present in various biological fluids. Generally, MSIA kits consist of devices, methods and reagents that facilitated the rapid and efficient extraction specific proteins and variants present in various biological fluids. Specifically, MSIA kits may consist of any or all of following items: MSIA-Tips, sample facilitating devices, samples, sample retaining/containment devices, activating reagents, affinity ligands, internal reference standards, buffers, rinse reagents, elution reagents, stabilizing reagents, mass spectrometry reagents and calibrants, mass spectrometry targets, mass spectrometers, analysis software, protein databases, instructional methods, specialized packaging and the like.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. A kit for isolating and qualitatively characterizing at least one target biomolecule in a plurality of biological fluids from a same individual or in a same type biological fluid of a plurality of individuals, said kit comprising:
at least one MSIA-Tip for each biological fluid of said same individual or at least one MSIA-Tip for each individual having said same type biological fluid for analysis, said MSIA-Tip having an affinity reagent comprising an affinity ligand for said at least one target biomolecule present within the tip; and
at least one mass spectrometer target.

2. The kit of claim 1 wherein said biomolecule comprises a protein.

3. The kit of claim 2 wherein said protein comprises at least one of urinary protein 1, IgG light chains kappa and lambda, insulin-like growth factor, serum amyloid, vitamin D binding protein, leptin, Tamm Horsfall Glycoprotein, albumin, lysozyme, a-defensins, immunoglobulin, apolipoprotein E, apolipoprotein AII, apolipoprotein Al, c-reactive protein, serum amyloid P component, cystatin C, transthyretin, transferring, and retinol binding protein.

4. The kit of claim 1 wherein said affinity ligand is anti-cystatin C antibody.

5. A kit for isolating and qualitatively characterizing at least one target biomolecule in a plurality of biological fluids from a same individual or in a same type biological fluid of a plurality of individuals, said kit comprising:
at least one MSIA-Tip for each biological fluid of said same individual or at least one MSIA-Tip for each individual having said same type biological fluid for analysis, said MSIA-Tip having an affinity reagent comprising an affinity ligand for said at least one target biomolecule present within the tip;
at least one internal reference standard of predetermined concentration in an amount sufficient to add said internal reference standard to each if said biological fluids of said same individual or said biological fluid of each of said plurality of individuals; and
at least one mass spectrometer target.

6. The kit of claim 5 wherein said biomolecule comprises a protein.

7. The kit of claim 6 wherein said protein comprises at least one of urinary protein 1, IgG light chains kappa and lambda, insulin-like growth factor, serum amyloid, vitamin D binding protein, leptin, Tamm Horsfall Glycoprotein, albumin, lysozyme, a-defensins, immunoglobulin, apolipoprotein E, apolipoprotein AII, apolipoprotein Al, c-reactive protein, serum amyloid P component, cystatin C, transthyretin, transferring, and retinol binding protein.

8. The kit of claim 5 wherein said affinity ligand is anti-cystatin C antibody.

9. The kit of claim 5 wherein said at least one internal reference standard is an internal reference standard that shares sequence homology with said at least one target biomolecule.

10. The kit of claim 9 wherein said at least one internal reference standard that shares sequence homology with said at least one biomolecule is selected from the group comprising enzymatic/chemically modified versions of said at least one target biomolecule, truncated/extended recombinant forms of said at least one target biomolecule, said at least one target biomolecule recombinantly expressed in isotopically-enriched media, and said at least one target biomolecule from a different biological species.
